SEQUENCE LISTING (37 sequences)

SEQ ID NO: 1 - DNA, 2346 bp, Artificial Sequence
  pTMH
gaattctaatacgactcactataggaccaagagacgggcccatatcgattcgcgaccgcgggatactagtcgtacgcctaggggtctcttggtgggtcggcatggcatctccacctcctcgcggtccgacctgggcatccgaaggaggacgtcgtccactcggatggctaagggagagctcggatccggctgctaacaaagcccgaaaggaagctgagttggctgctgccaccgctgagcaataactagcataaccccttggggcctctaaacgggtcttgaggggttttttgcatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 2 - DNA, 15192 bp, Artificial Sequence
  CND-745T
accaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactccgcacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagccctcaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccgcctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcagacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccactgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagaaggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgcgtacgggtagaagagtctggatcccgaccggcacattcaggacgcaatatgggctccaaactttctaccaggattccagcacctctgatgctgaccacccggattacgctgatattgagctgtatccgtccgacaagctctcttgacggcaggcctcttgcagctgcaggaattgtagtaacaggagataaggcagtcaatgtatacacctcgtctcagacagggtcaatcatagtcaagttgctcccgaatatgcccagggataaagaggcgtgtgcaaaagccccattagaggcatataacagaacactgactactttgctaactcctcttggcgactccatccgcaagatccaagggtctgtgtccacgtctggaggaggcagacaagcacgcctgataggtgctgttattggcagtgtagctcttggggttgcaacagcggcacagataacagcagcggccgccctaatacaagccaaccagaatgccgccaacatcctccggcttaaggagagcattgctgcaaccaatgaagctgtgcatgaagtcaccgacggattatcacaactatcagtggcagttgggaagatgcagcagttcgtcaatgaccagtttaataatacagcacgagaattggactgtataaaaatcacacaacaggttggtgtagagctaaacctatacctaactgaattgactacagtattcgggccacagatcacttcccctgcattaactcagttgaccatccaagcactttataatttagctggtggcaatatgaattacttattaactaagttaggtatagggaacaatcaactcagctcattaattggtagcggcctgatcactggttaccctatactgtatgattcacagactcaactcttgggcatacaagtgaatttgccctcagtcgggaacttaaataatatgcgtgccacctatttggagaccttatctgtaagtacaaccaaaggatatgcctcagcacttgtcccgaaagtagtgacacaggtcggttctgtgatagaagagctcgacacctcatactgcatagagtccgatctggatttatattgtactagaatagtgacattccccatgtccccaggtatttattcctgcttgagcggcaacacatcagcttgcatgtattcaaagactgaaggcgcactcactacgccgtatatggcccttaaaggctcggttattgccaattgtaagataacaacatgtagatgtacagaccctcctggtatcatatcgcaaaattatggagaagccgtatccctgatagatagacattcgtgcaatgtcttatcattagacgggataactctgaggctcagtggggaatttgatgcaacttatcaaaagaacatctcaatactagattctcaagtcatcgtgacaggcaatcttgatatctcaactgaacttggaaacgtcaacaattcaatcagcaatgccttggatagtttggcagaaagcaacagcaagctggaaaaaatcaatgtcagactaaccagcacatctgctctcattacctatattgttctaactgtcatttctctagttttcggtgcatttagtttgggtttagcgtgttacctgatgtacaaacagaaggcacaacaaaagaccttgctatggcttgggaataataccctcgatcagatgagagccactacaagagcatgaatgcagataagaggtgggtatatacccaacagcagcctgtgtatcaattccgataacctgtcaagtagaagacttaagaaaaaactactgggaataagcaaccaaagagcactacacgggtagaacgatcagaggagccacccttcaatcggaaattaggcttcacaacatccgttctaccgcatcaccaacaacaagagtcaatcatggaccgcgcggttaacagagtcgtgctggagaatgaggaaagagaagcaaagaacacatggcgcctggttttccggatcgcagttttacttttaatggtaatgactctagctatctcctcagctgccctggcatacagcacgggggccagtacgccgcacgacctcgcaagcatattgactgtgatctccaagacagaagataaggttacgtctttactcagttcaagtcaagacgtgatagataggatatacaagcaggtggctcttgaatccccgctggcactactaaacactgaatctgtaattatgaatgcaataacctctctttcttatcaaattaacggggctgcgaacaatagcggatgtggggcgcctgttcatgacccagattatatcggggggataggcaaagaactcatagtggacgacatcagtgatgttacatcattttatccttctgcatatcaagaacacttgaatttcatcccggcacctactacaggatccggttgcactcggataccctcgtttgacatgagcaccacccattattgttatactcacaatgtgatactatccggttgcagagatcactcacactcacatcaatacttagcacttggtgtgcttcggacatctgcaacagggagggtattcttttctactctgcgctctatcaatttagatgacacccaaaatcggaagtcctgcagtgtgagtgcaacccctttaggttgtgatatgctgtgctccaaggtcacagggactgaagaggaggattacaagtcagttgcccccacatcaatggtgcacggaaggctagggtttgacggtcaataccatgaaaaggacttagacaccacggtcttatttaaggattgggtggcaaattacccaggagcgggaggagggtcttttattgacgaccgtgtatggttcccagtttacggagggctcaaacccgattcacccagtgacactgcacaagaagggaaatacgtaatatacaagcgccataacaacacatgccccgataaacaagattaccaaattcggaaggctaagtcttcatataaacccgggcgatttggtgggaagcgcgtacagcaagccatcttatccatcaaagtgtcaacatctttgggtaaggacccggtgctgactattccacctaatacaatcacactcatgggagccgaaggcagaattctcacagtggggacatctcacttcttgtaccaacgagggtcttcatatttctcccctgccttattatatcccatgacagtaaataacaaaacggctacactccatagtccttatacgtttaatgctttcactcggccaggtagtgtcccttgccaggcatcagcaagatgccccaactcatgcattactggagtctatactgatccatatcccttaatcttccataggaatcatactctacgaggggtcttcggaacgatgcttgatgatgaacaagcgagacttaaccccgtatccgcagtattcgacaacgtatcccgcagtcgtgtcacccgggtgagttcaagcagcaccaaggcagcatacacgacatcgacatgtttcaaagttgtcaagaccaataaaacttattgtcttagtattgcagaaatatccaataccctgttcggggaatttaggatcgttcccttactagttgagatcctcaaggatgacggggttagagaagccaggtctggctagttgagtcaattataaaggagttggaaagatggcattgtatcacctatcttctgtgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggtaaataatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgtcgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttaagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagcaagatcgcattgtcgtgttacctgcatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggccgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggagtagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgattgttcttaccaactctattacctgagagtaagaggcctagacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatcccatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagtgacctgacctcagataaagcagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcaaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggt

SEQ ID NO: 3 - protein, 489 aa, Artificial Sequence
  CND-745T NP protein
MSSVFDEYEQLLAAQTRPNGAHGGGEKGSTLKVDVPVFTLNSDDPEDRWSFVVFCLRIAVSEDANKPLRQGALISLLCSHSQVMRNHVALAGKQNEATLAVLEIDGFANGTPQFNNRSGVSEERAQRFAMIAGSLPRACSNGTPFVTAGAEDDAPEDITDTLERILSIQAQVWVTVAKAMTAYETADESETRRINKYMQQGRVQKKYILYPVCRSTIQLTIRQSLAVRIFLVSELKRGRNTAGGTSTYYNLVGDVDSYIRNTGLTAFFLTLKYGINTKTSALALSSLSGDIQKMKQLMRLYRMKGDNAPYMTLLGDSDQMSFAPAEYAQLYSFAMGMASVLDKGTGKYQFARDFMSTSFWRLGVEYAQAQGSSINEDMAAELKLTPAARRGLAAAAQRVSEETSSIDMPTQQVGVLTGLSEGGSQALQGGSNRSQGQPEAGDGETQFLDLMRAVANSMREAPNSAQGTPQSGPPPTPGPSQDNDTDWGY

SEQ ID NO: 4 - protein, 395 aa, Artificial Sequence
  CND-745T P protein
MATFTDAEIDELFETSGTVIDNIITAQGKPAETVGRSAIPQGKTKVLSAAWEKHGSIQPPASQDNPDRQDRSDKQPSTPEQTTPHDSPPATSADQPPTQATDEAVDTQLRTGASNSLLLMLDKLSNKSSNAKKGPWSSPQEGNHQRPTQQQGSQPSRGNSQERPQNQVKAAPGNQGTDVNTAYHGQWEESQLSAGATPHALRSRQSQDNTLVSADHVQPPVDFVQAMMSMMEAISQRVSKVDYQLDLVLKQTSSIPMMRSEIQQLKTSVAVMEANLGMMKILDPGCANISSLSDLRAVARSHPVLVSGPGDPSPYVTQGGEMALNKLSQPVPHPSELIKPATACGPDIGVEKDTVRALIMSRPMHPSSSAKLLSKLDAAGSIEEIRKIKRLALNG

SEQ ID NO: 5 - protein, 364 aa, Artificial Sequence
  CND-745T M protein
MDSSRTIGLYFDSAHSSSNLLAFPIVLQDTGDGKKQIAPQYRIQRLDLWTDSKEDSVFITTYGFIFQVGNEEATVGMIDDKPKRELLSAAMLCLGSVPNTGDLIELARACLTMIVTCKKSATNTERMVFSVVQAPQVLQSCRVVANKYSSVNAVKHVKAPEKIPGSGTLEYKVNFVSLTVVPKKDVYKIPAAVLKVSGSSLYNLALNVTINVEVDPRSPLVKSLSKSDSGYYANLFLHIGLMTTVDRKGKKVTFDKLEKKIRSLDLSVGLSDVLGPSVLVKARGARTKLLAPFFSSSGTACYPIANASPQVAKILWSQTACLRSVKIIIQAGTQRAVAVTADHEVTSTKLEKGHTLAKYNPFKK

SEQ ID NO: 6 - protein, 553 aa, Artificial Sequence
  CND-745T F protein
MGSKLSTRIPAPLMLTTRITLILSCIRPTSSLDGRPLAAAGIVVTGDKAVNVYTSSQTGSIIVKLLPNMPRDKEACAKAPLEAYNRTLTTLLTPLGDSIRKIQGSVSTSGGGRQARLIGAVIGSVALGVATAAQITAAAALIQANQNAANILRLKESIAATNEAVHEVTDGLSQLSVAVGKMQQFVNDQFNNTARELDCIKITQQVGVELNLYLTELTTVFGPQITSPALTQLTIQALYNLAGGNMNYLLTKLGIGNNQLSSLIGSGLITGYPILYDSQTQLLGIQVNLPSVGNLNNMRATYLETLSVSTTKGYASALVPKVVTQVGSVIEELDTSYCIESDLDLYCTRIVTFPMSPGIYSCLSGNTSACMYSKTEGALTTPYMALKGSVIANCKITTCRCTDPPGIISQNYGEAVSLIDRHSCNVLSLDGITLRLSGEFDATYQKNISILDSQVIVTGNLDISTELGNVNNSISNALDSLAESNSKLEKINVRLTSTSALITYIVLTVISLVFGAFSLGLACYLMYKQKAQQKTLLWLGNNTLDQMRATTRA

SEQ ID NO: 7 - protein, 577 aa, Artificial Sequence
  CND-745T HN protein
MDRAVNRVVLENEEREAKNTWRLVFRIAVLLLMVMTLAISSAALAYSTGASTPHDLASILTVISKTEDKVTSLLSSSQDVIDRIYKQVALESPLALLNTESVIMNAITSLSYQINGAANNSGCGAPVHDPDYIGGIGKELIVDDISDVTSFYPSAYQEHLNFIPAPTTGSGCTRIPSFDMSTTHYCYTHNVILSGCRDHSHSHQYLALGVLRTSATGRVFFSTLRSINLDDTQNRKSCSVSATPLGCDMLCSKVTGTEEEDYKSVAPTSMVHGRLGFDGQYHEKDLDTTVLFKDWVANYPGAGGGSFIDDRVWFPVYGGLKPDSPSDTAQEGKYVIYKRHNNTCPDKQDYQIRKAKSSYKPGRFGGKRVQQAILSIKVSTSLGKDPVLTIPPNTITLMGAEGRILTVGTSHFLYQRGSSYFSPALLYPMTVNNKTATLHSPYTFNAFTRPGSVPCQASARCPNSCITGVYTDPYPLIFHRNHTLRGVFGTMLDDEQARLNPVSAVFDNVSRSRVTRVSSSSTKAAYTTSTCFKVVKTNKTYCLSIAEISNTLFGEFRIVPLLVEILKDDGVREARSG

SEQ ID NO: 8 - protein, 2204 aa, Artificial Sequence
  CND-745T L protein
MASSGPERAEHQIILPESHLSSPLVKHKLLYYWKLTGLPLPDECDFDHLILSRQWKKILESASPDTERMIKLGRAVHQTLNHNSRITGVLHPRCLEELANIEVPDSTNKFRKIEKKIQIHNTRYGELFTRLCTHIEKKLLGSSWSNNVPRSEEFSSIRTDPAFWFHSKWSTAKFAWLHIKQIQRHLMVAARTRSAANKLVMLTHKVGQVFVTPELVVVTHTNENKFTCLTQELVLMYADMMEGRDMVNIISTTAVHLRSLSEKIDDILRLIDALAKDLGNQVYDVVSLMEGFAYGAVQLLEPSGTFAGDFFAFNLQELKDILIGLLPNDIAESVTHAIATVFSGLEQNQAAEMLCLLRLWGHPLLESRIAAKAVRSQMCAPKMVDFDMILQVLSFFKGTIINGYRKKNAGVWPRVKVDTIYGKVIGQLHADSAEISHDIMLREYKSLSALEFEPCIEYDPVTNLSMFLKDKAIAHPNDNWLASFRRNLLSEDQKKHVKEATSTNRLLIEFLESNDFDPYKEMEYLTTLEYLRDDNVAVSYSLKEKEVKVNGRIFAKLTKKLRNCQVMAEGILADQIAPFFQGNGVIQDSISLTKSMLAMSQLSFNSNKKRITDCKERVSSNRNHDPKSKNRRRVATFITTDLQKYCLNWRYQTIKLFAHAINQLMGLPHFFEWIHLRLMDTTMFVGDPFNPPSDPTDCDLSRVPNDDIYIVSARGGIEGLCQKLWTMISIAAIQLAAARSHCRVTCMVQGDNQVIAVTREVRSDDSPEMVLTQLHQASDNFFKELIHVNHLIGHNLKDRETIRSDTFFIYSKRIFKDGAILSQVLKNSSKLVLVSGDLSENTVMSCANIASTVARLCENGLPKDFCYYLNYIMSCVQTYFDSEFSITNNSHPDLNQSWIEDISFVHSYVLTPAQLGGLSNLQYSRLYTRNIGDPGTTAFAEIKRLEAVGLLSPNIMTNILTRPPGNGDWASLCNDPYSFNFETVASPNIVLKKHTQRVLFETCSNPLLSGVHTEDNEAEEKALAEFLLNQEVIHPRVAHAIMEASSVGRRKQIQGLVDTTNTVIKIALTRRPLGIKRLMRIVNYSSMHAMLFRDDVFSSSRSNHPLVSSNMCSLTLADYARSRSWSPLTGGRKILGVSNPDTIELVEGEILSVSGGCTRCDSGDEQFTWFHLPSNIELTDDTSKNPPMRVPYLGSKTQERRAASLAKIAHMSPHVKAALRASSVLIWAYGDNEVNWTAALTIAKSRCNVNLEYLRLLSPLPTAGNLQHRLDDGITQMTFTPASLYRVSPYIHISNDSQRLFTEEGVKEGNVVYQQIMLLGLSLIESIFPMTTTRTYDEITLHLHSKFSCCIREAPVAVPFELLGVVPELRTVTSNKFMYDPSPVSEGDFARLDLAIFKSYELNLESYPTIELMNILSISSGKLIGQSVVSYDEDTSIKNDAIIVYDNTRNWISEAQNSDVVRLFEYAALEVLLDCSYQLYYLRVRGLDNIVLYMGDLYKNMPGILLSNIAATISHPVIHSRLHAVGLVNHDGSHQLADTDFIEMSAKLLVSCTRRVISGLYSGNKYDLLFPSVLDDNLNEKMLQLISRLCCLYTVLFATTREIPKIRGLTAEEKCSILTEYLLSDAVKPLLSPDQVSSIMSPNIITFPANLYYMSRKSLNLIREREDRDTILALLFPQEPLLEFPSVQDIGARVKDPFTRQPAAFLQELDLSAPARYDAFTLSQIHPELTSPNPEEDYLVRYLFRGIGTASSSWYKASHLLSVPEVRCARHGNSLYLAEGSGAIMSLLELHVPHETIYYNTLFSNEMNPPQRHFGPTPTQFLNSVVYRNLQAEVTCKDGFVQEFRPLWRENTEESDLTSDKAVGYITSAVPYRSVSLLHCDIEIPPGSNQSLLDQLAINLSLIAMHSVREGGVVIIKVLYAMGYYFHLLMNLFAPCSTKGYILSNGYACRGDMECYLVFVMGYLGGPTFVHEVVRMAKTLVQRHGTLLSKSDEITLTRLFTSQRQRVTDILSSPLPRLIKYLRKNIDTALIEAGGQPVRPFCAESLVSTLANITQITQIIASHIDTVIRSVIYMEAEGDLADTVFLFTPYNLSTDGKKRTSLKQCTRQILEVTILGLRVENLNKIGDIISLVLKGMISMEDLIPLRTYLKHSTCPKYLKAVLGITKLKEMFTDTSVLYLTRAQQKFYMKTIGNAVKGYYSNCDS

SEQ ID NO: 9 - protein, 2204 aa, Artificial Sequence
  KBNP-C4152 L protein
MASSGPERAEHQIILPESHLSSPLVKHKLLYYWKLTGLPLPDECDFDHLILSRQWKKILESASPDTERMIKLGRAVHQTLNHNSRITGVLHPRCLEELANIEVPDSTNKFRKIEKKIQIHNTRYGELFTRLCTHIEKKLLGSSWSNNVPRSEEFSSIRTDPAFWFHSKWSTAKFAWLHIKQIQRHLMVAARTRSAANKLVMLTHKVGQVFVTPELVVVTHTNENKFTCLTQELVLMYADMMEGRDMVNIISTTAVHLRSLSEKIDDILRLIDALAKDLGNQVYDVVSLMEGFAYGAVQLLEPSGTFAGDFFAFNLQELKDILIGLLPNDIAESVTHAIATVFSGLEQNQAAEMLCLLRLWGHPLLESRIAAKAVRSQMCAPKMVDFDMILQVLSFFKGTIINGYRKKNAGVWPRVKVDTIYGKVIGQLHADSAEISHDIMLREYKSLSALEFEPCIEYDPVTNLSMFLKDKAIAHPNDNWLASFRRNLLSEDQKKHVKEATSTNRLLIEFLESNDFDPYKEMEYLTTLEYLRDDNVAVSYSLKEKEVKVNGRIFAKLTKKLRNCQVMAEGILADQIAPFFQGNGVIQDSISLTKSMLAMSQLSFNSNKKRITDCKERVSSNRNHDPKSKNRRRVATFITTDLQKYCLNWRYQTIKLFAHAINQLMGLPHFFEWIHLRLMDTTMFVGDPFNPPSDPTDCDLSRVPNDDIYIVSARGGIEGLCQKLWTMISIAAIQLAAARSHCRVACMVQGDNQVIAVTREVRSDDSPEMVLTQLHQASDNFFKELIHVNHLIGHNLKDRETIRSDTFFIYSKRIFKDGAILSQVLKNSSKLVLVSGDLSENTVMSCANIASTVARLCENGLPKDFCYYLNYIMSCVQTYFDSEFSITNNSHPDLNQSWIEDISFVHSYVLTPAQLGGLSNLQYSRLYTRNIGDPGTTAFAEIKRLEAVGLLSPNIMTNILTRPPGNGDWASLCNDPYSFNFETVASPNIVLKKHTQRVLFETCSNPLLSGVHTEDNEAEEKALAEFLLNQEVIHPRVAHAIMEASSVGRRKQIQGLVDTTNTVIKIALTRRPLGIKRLMRIVNYSSMHAMLFRDDVFSSSRSNHPLVSSNMCSLTLADYARSRSWSPLTGGRKILGVSNPDTIELVEGEILSVSGGCTRCDSGDEQFTWFHLPSNIELTDDTSKNPPMRVPYLGSKTQERRAASLAKIAHMSPHVKAALRASSVLIWAYGDNEVNWTAALTIAKSRCNVNLEYLRLLSPLPTAGNLQHRLDDGITQMTFTPASLYRVSPYIHISNDSQRLFTEEGVKEGNVVYQQIMLLGLSLIESIFPMTTTRTYDEITLHLHSKFSCCIREAPVAVPFELLGVVPELRTVTSNKFMYDPSPVSEGDFARLDLAIFKSYELNLESYPTIELMNILSISSGKLIGQSVVSYDEDTSIKNDAIIVYDNTRNWISEAQNSDVVRLFEYAALEVLLDCSYQLYYLRVRGLDNIVLYMGDLYKNMPGILLSNIAATISHPVIHSRLHAVGLVNHDGSHQLADTDFIEMSAKLLVSCTRRVISGLYSGNKYDLLFPSVLDDNLNEKMLQLISRLCCLYTVLFATTREIPKIRGLTAEEKCSILTEYLLSDAVKPLLSPDQVSSIMSPNIITFPANLYYMSRKSLNLIREREDRDTILALLFPQEPLLEFPSVQDIGARVKDPFTRQPAAFLQELDLSAPARYDAFTLSQIHPELTSPNPEEDYLVRYLFRGIGTASSSWYKASHLLSVPEVRCARHGNSLYLAEGSGAIMSLLELHVPHETIYYNTLFSNEMNPPQRHFGPTPTQFLNSVVYRNLQAEVTCKDGFVQEFRPLWRENTEESDLTSDKAVGYITSAVPYRSVSLLHCDIEIPPGSNQSLLDQLAINLSLIAMHSVREGGVVIIKVLYAMGYYFHLLMNLFAPCSTKGYILSNGYACRGDMECYLVFVMGYLGGPTFVHEVVRMAKTLVQRHGTLLSKSDEITLTRLFTSQRQRVTDILSSPLPRLIKYLRKNIDTALIEAGGQPVRPFCAESLVSTLANITQITQIIASHIDTVIRSVIYMEAEGDLADTVFLFTPYNLSTDGKKRTSLKQCTRQILEVTILGLRVENLNKIGDIISLVLKGMISMEDLIPLRTYLKHSTCPKYLKAVLGITKLKEMFTDTSVLYLTRAQQKFYMKTIGNAVKGYYSNCDS

SEQ ID NO: 10 - DNA, 6 bp, Artificial Sequence
  Mlu I site
acgcgt

SEQ ID NO: 11 - DNA, 19 bp, Artificial Sequence
  NDPt-F
ggaaggagacrraaacgct

SEQ ID NO: 12 - DNA, 20 bp, Artificial Sequence
  NDPt-R
tgccactgmtagttgygata

SEQ ID NO: 13 - DNA, 20 bp, Artificial Sequence
  NDcomF156
atacacctcrtcycagacag

SEQ ID NO: 14 - DNA, 21 bp, Artificial Sequence
  NDV-L gene-10568-F
tgacatatatattgtcagtgc

SEQ ID NO: 15 - DNA, 19 bp, Artificial Sequence
  NDV-L gene-3673-R
acacggatgatgcccttag

SEQ ID NO: 16 - DNA, 19 bp, Artificial Sequence
  Lasota-P1129F
gatgcagccgggtcgatcg

SEQ ID NO: 17 - DNA, 19 bp, Artificial Sequence
  LasotaNDVC7d-Fgene-904R
aggtggcacgcatattatt

SEQ ID NO: 18 - DNA, 20 bp, Artificial Sequence
  NDcom156/f
atacacctcrtcycagacag

SEQ ID NO: 19 - DNA, 22 bp, Artificial Sequence
  La6203R
acatttttgtagtggcyctcat

SEQ ID NO: 20 - DNA, 19 bp, Artificial Sequence
  NDV C7d-F-5704-F
tgagcggcaacacatcagc

SEQ ID NO: 21 - DNA, 21 bp, Artificial Sequence
  SF-7575R
ttaggtggaatagtcagcacc

SEQ ID NO: 22 - DNA, 19 bp, Artificial Sequence
  NDV-all-HN-737F
ttgtgatatgctgtgctct

SEQ ID NO: 23 - DNA, 18 bp, Artificial Sequence
  NDV--HN-L-intetgenicR
aagataggtgatacaatg

SEQ ID NO: 24 - DNA, 19 bp, Artificial Sequence
  NDV C7d-HN-7834-F
aggtagtgtcccttgccag

SEQ ID NO: 25 - DNA, 18 bp, Artificial Sequence
  NDV-All-9573-R
tgcgcacatttggctcct

SEQ ID NO: 26 - DNA, 21 bp, Artificial Sequence
  NDV-All-9375-F
gatttcttcgcattcaacctg

SEQ ID NO: 27 - DNA, 20 bp, Artificial Sequence
  NDV-All-10644-R
gctgcagcaagttggattgc

SEQ ID NO: 28 - DNA, 20 bp, Artificial Sequence
  NDV-All-10568-F
tgacatatatattgtcagtg

SEQ ID NO: 29 - DNA, 19 bp, Artificial Sequence
  NDV C7dCND-L-3673R
acacggatgatgcccttag

SEQ ID NO: 30 - DNA, 19 bp, Artificial Sequence
  NDV C7dCND-L-3512F
atcttccaagcaatataga

SEQ ID NO: 31 - DNA, 17 bp, Artificial Sequence
  NDV C7dCND-L-5373R
gatgccttataccaaga

SEQ ID NO: 32 - DNA, 19 bp, Artificial Sequence
  NDV C7dCND-L-5068F
attggtgctcgagtgaaag

SEQ ID NO: 33 - DNA, 20 bp, Artificial Sequence
  CND-Trailer-37R
gagttcgaattcgagtccta

SEQ ID NO: 34 - DNA, 35 bp, Artificial Sequence
  CND-Lgene-A745-F
tatgctagcgatgagtcaactgtcttttaacagca

SEQ ID NO: 35 - DNA, 35 bp, Artificial Sequence
  CND-Lgene-A745-R
tcgctagcgtgctcaccagactctccgcacagaat

SEQ ID NO: 36 - DNA, 36 bp, Artificial Sequence
  CND-Lgene-A745-IF
atcgcattgtcgtgttacctgcatggtacagggtga

SEQ ID NO: 37 - DNA, 36 bp, Artificial Sequence
  CND-Lgene-A745-IR
tcaccctgtaccatgcaggtaacacgacaatgcgat

(19) United States
(12) Patent Application Publication
(10) Pub. No.: US 2026/0061047 A1
(43) Pub. Date: Mar. 5, 2026

(54) Newcastle Disease Virus with Improved Heat Resistance, and Newcastle Disease Virus Vaccine Comprising Same

(71) Applicant: BIOPOA, INC., Hwaseong-si, Gyeonggi-do (KR)

(72) Inventors: Sun-Hee Cho, Seongnam-si, Gyeonggi-do (KR); Tae-Eun Kim, Yongin-si, Gyeonggi-do (KR)

(21) Appl. No.: 19/125,251

(22) PCT Filed: Feb. 8, 2022
(86) PCT No.: PCT/KR2022/001899

§ 371 Date: Apr. 28, 2025

(30) Foreign Application Priority Data
Feb. 8, 2021 (KR) 10-2021-0017203

(51) Int. Cl. A61K 39/17; A61K 39/00; A61P 37/04; C12N 7/00

(52) U.S. Cl. CPC A61K 39/17; A61P 37/04; C12N 7/00; A61K 2039/545; C12N 2760/18121; C12N 2760/18134; C12N 2760/18152; C12N 2760/18162; C12N 2760/18171

(57) ABSTRACT

The present specification provides: a Newcastle disease virus with improved heat resistance; a Newcastle disease vaccine comprising the virus; a polypeptide comprising an L protein in the virus; a polynucleotide encoding same; and a recombinant vector comprising the polynucleotide.

### TECHNICAL FIELD

The present disclosure provides a Newcastle disease virus with improved thermostability, a Newcastle disease virus vaccine comprising the virus, a polypeptide comprising the L protein in the virus, a polynucleotide encoding the polypeptide, and a recombinant vector comprising the polynucleotide.

### REFERENCE TO AN ELECTRONIC SEQUENCE LISTING

The contents of the electronic sequence listing (JUNG-070_Sequence_Listing.txt; Size: 83,605 bytes; and Date of Creation: Jun. 10, 2025) is herein incorporated by reference in its entirety.

### BACKGROUND ART

Newcastle disease (ND) is one of the fifteen most important livestock diseases worldwide. It is an acute febrile respiratory disease and a legally designated Class I infectious disease, with a mortality rate of 100% in non-immunized poultry. The Republic of Korea is considered an endemic region for Newcastle disease virus (NDV), and eradication of the disease is expected to present significant challenges.

Newcastle disease virus is classified into two classes based on the nucleotide sequence of the F gene. Class I possesses only a single genotype, is primarily isolated from wild birds, and is generally lentogenic. Class II includes at least 18 genotypes (genotypes I to XVIII) identified to date and is categorized into lentogenic, mesogenic, and velogenic strains depending on pathogenicity. Genotypes I and II of Class II are classified as lentogenic strains, among which the VG/GA strain (Avinew) and the LaSota strain (including the Clone 30 strain) are well known as representative live vaccine strains for Newcastle disease that have been most widely used both in Korea and worldwide. However, genotype VII, a velogenic strain responsible for recent global outbreaks, exhibits considerable antigenic differences from the lentogenic strains of genotypes I and II. Accordingly, there has been a need for vaccines based on genotypes more closely related to those of velogenic field strains. To address this issue, the research team at Biopoa Co., Ltd. developed, for the first time in the world, a genotype VII virus with the pathogenicity removed, KBNP-C4152R2L (N+), using reverse genetics techniques [2,3,4]. In this regard, reference is made to the following documents, the entire contents of which are incorporated herein by reference:
1. Korean Patent No. 10-0801180-00-00
2. Korean Patent No. 10-0862049-00-00

However, the KBNP-C4152R2L (N+) virus is thermolabile; therefore, improvement in the thermostability of the vaccine strain is required for its use as a highly stable vaccine.

### DETAILED DESCRIPTION OF THE INVENTION

### Technical Problem

For improved thermostability, the inventors of the present invention developed a thermostable Newcastle disease virus (NDV) by modifying a specific amino acid of the L protein of the KBNP-C4152 virus (Accession No.: KCTC 10984BP), which has been verified for safety and efficacy both domestically and internationally for over ten years. The improvement in thermostability was confirmed through experimentation.

It is an object of the present disclosure to provide a Newcastle disease virus in which the 745^{th} amino acid of the L protein is mutated to an amino acid other than alanine.

In one example, the amino acid other than alanine in the Newcastle disease virus may be threonine (T), isoleucine (I), leucine (L), lysine (K), methionine (M), phenylalanine (F), tryptophan (W), valine (V), histidine (H), arginine (R), asparagine (N), aspartic acid (D), cysteine (C), selenocysteine (U), glutamic acid (E), glutamine (Q), glycine (G), proline (P), serine (S), or tyrosine (Y).

The Newcastle disease virus in which the 745^{th} amino acid of the L protein is mutated may exhibit improved thermostability compared to a Newcastle disease virus in which the 745^{th} amino acid is alanine. The Newcastle disease virus with improved thermostability as described above may be used as a vaccine strain applicable to a Newcastle disease virus vaccine.

Another object of the present disclosure is to provide a Newcastle disease virus vaccine comprising the Newcastle disease virus.

Still another object of the present disclosure is to provide a polypeptide comprising an amino acid sequence in which an amino acid residue corresponding to the 745^{th} amino acid of the L protein of SEQ ID NO: 9 is substituted with an amino acid other than alanine, the polypeptide being capable of enhancing viral thermostability.

Yet another object of the present disclosure is to provide a polypeptide, which is an L protein mutant, comprising an amino acid sequence in which an amino acid residue corresponding to the 745^{th} amino acid of the L protein of SEQ ID NO: 9 is substituted with an amino acid other than alanine, the polypeptide being capable of enhancing viral thermostability.

The amino acid other than alanine may be threonine (T), isoleucine (I), leucine (L), lysine (K), methionine (M), phenylalanine (F), tryptophan (W), valine (V), histidine (H), arginine (R), asparagine (N), aspartic acid (D), cysteine (C), selenocysteine (U), glutamic acid (E), glutamine (Q), glycine (G), proline (P), serine (S), or tyrosine (Y), but is not limited thereto.

Still yet another object of the present disclosure is to provide a polypeptide comprising the amino acid sequence of SEQ ID NO: 8 and capable of enhancing viral thermostability.

A further object of the present disclosure is to provide a polynucleotide encoding the polypeptide and a recombinant vector comprising the polynucleotide.

Another further object of the present disclosure is to provide a composition for immunization against Newcastle disease virus, and/or for prevention, treatment, and/or amelioration of Newcastle disease, the composition comprising the Newcastle disease virus, a Newcastle disease vaccine composition, the polypeptide, the polynucleotide, and/or the recombinant vector.

A still further object of the present disclosure is to provide a method for immunization against Newcastle disease virus, and/or for prevention, treatment, and/or amelioration of Newcastle disease, the method comprising the step of administering the Newcastle disease virus, a Newcastle disease vaccine composition, the polypeptide, the polynucleotide, and/or the recombinant vector to a subject in need thereof.

Yet another further object of the present disclosure is to provide a method for producing a Newcastle disease virus with improved thermostability, the method comprising the step of inducing a mutation at the 745^{th} amino acid of the L protein to an amino acid other than alanine.

The L protein may comprise the amino acid sequence of SEQ ID NO: 9, and the step of inducing the mutation may comprise the step of inducing a mutation at the 745^{th} amino acid of the L protein of SEQ ID NO: 9, but is not limited thereto.

The amino acid other than alanine may be threonine (T), isoleucine (I), leucine (L), lysine (K), methionine (M), phenylalanine (F), tryptophan (W), valine (V), histidine (H), arginine (R), asparagine (N), aspartic acid (D), cysteine (C), selenocysteine (U), glutamic acid (E), glutamine (Q), glycine (G), proline (P), serine (S), or tyrosine (Y), but is not limited thereto.

### Technical Solution

The present disclosure provides a Newcastle disease virus comprising NP, P, M, L, F and HN proteins, wherein the 745^{th} amino acid of the L protein is an amino acid other than alanine, and the Newcastle disease virus exhibits improved thermostability compared to a Newcastle disease virus in which the 745^{th} amino acid is alanine.

The amino acid other than alanine may be threonine (T), isoleucine (I), leucine (L), lysine (K), methionine (M), phenylalanine (F), tryptophan (W), valine (V), histidine (H), arginine (R), asparagine (N), aspartic acid (D), cysteine (C), selenocysteine (U), glutamic acid (E), glutamine (Q), glycine (G), proline (P), serine (S), or tyrosine (Y), but is not limited thereto.

The present disclosure also discloses a Newcastle disease virus comprising:
NP, P. M and L proteins of a lentogenic Newcastle disease virus LaSota (AY845400); and
F and HN proteins of a velogenic Newcastle disease virus KBNP-4152 (Accession No.: KCTC 10919BP),
wherein the 115^{th} amino acid of the F protein of the velogenic Newcastle disease virus is an amino acid selected from the group consisting of alanine, aspartic acid, phenylalanine, isoleucine, leucine, serine, threonine, valine, and tyrosine,
wherein the 745^{th} amino acid of the L protein is mutated to an amino acid other than alanine, and
wherein the Newcastle disease virus exhibits improved thermostability compared to a Newcastle disease virus in which the 745^{th} amino acid is alanine.

The amino acid other than alanine may be threonine (T), isoleucine (I), leucine (L), lysine (K), methionine (M), phenylalanine (F), tryptophan (W), valine (V), histidine (H), arginine (R), asparagine (N), aspartic acid (D), cysteine (C), selenocysteine (U), glutamic acid (E), glutamine (Q), glycine (G), proline (P), serine (S), or tyrosine (Y), but is not limited thereto.

In one example, the L protein may be represented by (or may consist of or consist essentially of, or comprise) SEQ ID NO: 8, and the F protein may be represented by (or may consist of or consist essentially of, or comprise) SEQ ID NO: 6, but is not limited thereto.

The present disclosure also provides a Newcastle disease virus wherein the HN protein is a recombinant HN protein, in which an amino acid sequence downstream of position 570 of the HN protein of the lentogenic Newcastle disease virus strain LaSota (AY845400) is additionally inserted at the C-terminus of the 569^{th} amino acid of the HN protein of the velogenic Newcastle disease virus KBNP-4152 (Accession No.: KCTC 10919BP).

The present disclosure also provides a Newcastle disease virus comprising a genome represented by the nucleotide sequence of SEQ ID NO: 2.

The present disclosure also provides a Newcastle disease vaccine comprising the Newcastle disease virus.

The present disclosure also provides a polypeptide comprising an amino acid sequence in which an amino acid residue corresponding to the 745^{th} amino acid of the L protein of SEQ ID NO: 9 is substituted with an amino acid other than alanine, the polypeptide being capable of enhancing viral thermostability.

The amino acid other than alanine may be threonine (T), isoleucine (I), leucine (L), lysine (K), methionine (M), phenylalanine (F), tryptophan (W), valine (V), histidine (H), arginine (R), asparagine (N), aspartic acid (D), cysteine (C), selenocysteine (U), glutamic acid (E), glutamine (Q), glycine (G), proline (P), serine (S), or tyrosine (Y), but is not limited thereto.

The present disclosure also provides a polypeptide comprising the amino acid sequence of SEQ ID NO: 8 and capable of enhancing viral thermostability.

The present disclosure also provides a polynucleotide encoding the polypeptide.

The present disclosure also provides a recombinant vector comprising the polynucleotide.

The present disclosure also provides a composition for immunization against Newcastle disease virus, and/or for prevention, treatment, and/or amelioration of Newcastle disease, the composition comprising the Newcastle disease virus, a Newcastle disease vaccine composition, the polypeptide, the polynucleotide, and/or the recombinant vector.

The present disclosure also provides a method for immunization against Newcastle disease virus, and/or for prevention, treatment, and/or amelioration of Newcastle disease, the method comprising the step of administering the Newcastle disease virus, a Newcastle disease vaccine composition, the polypeptide, the polynucleotide, and/or the recombinant vector to a subject in need thereof.

The present disclosure also provides a method for producing a Newcastle disease virus with improved thermostability, the method comprising the step of inducing a mutation at the 745^{th} amino acid of the L protein to an amino acid other than alanine.

The amino acid other than alanine may be threonine (T), isoleucine (I), leucine (L), lysine (K), methionine (M), phenylalanine (F), tryptophan (W), valine (V), histidine (H), arginine (R), asparagine (N), aspartic acid (D), cysteine (C), selenocysteine (U), glutamic acid (E), glutamine (Q), glycine (G), proline (P), serine (S), or tyrosine (Y), but is not limited thereto.

Hereinafter, the present invention will be described in more detail.

In the present disclosure, when it is stated that a polynucleotide (which may be used interchangeably with the term “gene”) or a polypeptide (which may be used interchangeably with the term “protein”) “comprises a specific nucleic acid sequence or amino acid sequence” or “consists of or is represented by a specific nucleic acid sequence or amino acid sequence”, it may mean that the polynucleotide or polypeptide essentially includes the specific nucleic acid sequence or amino acid sequence. This may also be interpreted to include “a substantially equivalent sequence” in which the specific nucleic acid sequence or amino acid sequence has undergone a mutation (such as a deletion, substitution, modification, and/or addition), to the extent that the original and/or intended function of the polynucleotide or polypeptide is retained, and this does not exclude such a mutation.

In one example, when it is stated that a polynucleotide or polypeptide “comprises a specific nucleic acid sequence or amino acid sequence” or “consists of or is represented by a specific nucleic acid sequence or amino acid sequence”, it may mean that the polynucleotide or polypeptide (i) consists of the specific nucleic acid sequence or amino acid sequence, or (ii) comprises or consist essentially of a nucleic acid sequence or amino acid sequence having at least 96%, at least 97%, at least 98%, at least 99%, at least 99.5%, or at least 99.9% identity to the specific nucleic acid sequence or amino acid sequence, and retains its original and/or desired function. The term “identity” refers to the degree of match between a given nucleic acid sequence or amino acid sequence and another sequence, and may be expressed as a percentage (%). In the case of nucleic acid sequences, identity may be determined using algorithms such as BLAST (see Karlin and Altschul, Proc. Natl. Acad. Sci. USA, 90:5873, 1993) or FASTA (see Pearson, Methods Enzymol., 183:63, 1990), as described in the literature. Based on the BLAST algorithm, programs known as BLASTN and BLASTX have been developed (see http://www.ncbi.nlm.nih.gov).

As used herein, the terms “vaccine” and “vaccine composition” against Newcastle disease virus are used interchangeably to refer to a formulation comprising an immunogen, such as the Newcastle disease virus itself or any immunogenic fragment or fraction thereof, preferably an attenuated Newcastle disease virus, such as virus-like particles (VLPs) of the Newcastle disease virus with improved thermostability according to the present disclosure, which induces a cell-mediated and/or antibody-mediated immune response against Newcastle disease virus in a host. Such a formulation may also be referred to as an “immunogenic composition.” The vaccine composition may provide a protective immunity against Newcastle disease virus infection and/or associated clinical signs.

The “vaccine composition” may be a live vaccine, an inactivated vaccine, a subunit vaccine, a vector vaccine, a chimeric vaccine, or a DNA vaccine, but is not limited thereto. Moreover, the “vaccine composition” may be administered via an in ovo, intranasal, intratracheal, oral, intradermal, intramuscular, intraperitoneal, intravenous, conjunctival, or subcutaneous route, but is not limited thereto.

As used herein, the term “immune response” refers to any cell-mediated and/or antibody-mediated immune response against a chimeric virus or vaccine in an animal that has been administered the Newcastle disease virus with improved thermostability according to the present disclosure or a vaccine composition comprising the same. Typically, the “immune response” includes, but is not limited to, one or more of the following effects: the production or activation of antibodies, B cells, helper T cells, suppressor T cells, cytotoxic T cells, and/or γδ T cells, which are specifically induced against one or more antigens contained in the composition or vaccine. It is preferred that the host exhibits a therapeutic or protective immunological response, such that enhanced resistance to new infection and/or reduced clinical severity of disease is observed, as compared to a control group not administered the immunogenic composition or vaccine. Such prevention may be evidenced by at least the absence of symptoms associated with the above-described host infection and/or a reduction in the frequency or severity of such symptoms.

As used herein, the term “vaccinating” refers to the administration of a vaccine comprising virus-like particles (VLPs) of Newcastle disease virus, as described herein, prior to exposure to a Newcastle disease virus infectious disease.

As used herein, the term “prevent” or “prevention” refers to a reduction in the incidence, severity, or frequency of clinical signs of Newcastle disease virus infection as a result of administration of a vaccine composition comprising virus-like particles (VLPs) of the Newcastle disease virus with improved thermostability according to the present disclosure. The reduction in severity or frequency is determined in comparison to an animal or group of animals that has not been administered the vaccine composition of the present disclosure. In one embodiment, the animal may be a chicken.

The present disclosure provides a method for preventing Newcastle disease, the method comprising the step of administering the vaccine composition to an animal.

As used herein, the term “animal” may refer to poultry, including chickens, pheasants, ducks, geese, turkeys, and quails.

For convenience, the nucleic acid sequences described herein are represented based on DNA nucleotides. In the case where the polynucleotide is RNA, the sequence refers to one in which all or part of the thymine (T) residues in the nucleic acid sequence are substituted with uracil (U). Unless otherwise specified, the nucleic acid sequences described herein are shown in the 5′ to 3′ direction.

Unless otherwise specified herein, all temperatures are expressed in degrees Celsius.

LaSota, which is currently used as a vaccine strain, belongs to genotype II, whereas most of the strains currently identified as field strains are genetically distant, primarily belonging to genotypes VI and VII. For example, the 345-PDEQDYQIR-353 site in the HN protein of NDV is known to be a critical linear epitope involved in the induction of neutralizing antibodies. In Korea, pathogenic NDVs of genotypes VI (e.g., 95-98, 99-70, and 99-71) and VII have coexisted; however, NDVs of genotype VI have not been isolated at all from 2000 to 2006, with the last isolation occurring in 1999. Although NDVs of genotype VIIa were first isolated from poultry in 1995, they have not been detected thereafter, and only NDVs of genotype VIId have been isolated since then. For genotype VI viruses, a linear epitope mutation (E347K) was first identified in isolates obtained between 1993 and 1994 (SNU9358GG and SNU9444), and was consistently observed in subsequent isolates such as 95-98, 99-70, and 99-71. These observations suggest that such mutant strains may have temporarily evaded immune responses and persisted for a while. However, they are now considered to be nearly extinct following the nationwide spread of genotype VIId viruses after the year 2000. For genotype VII viruses, all isolates obtained between 1995 and 2001 exhibited linear epitopes identical to that of the LaSota strain. However, in 2002, a mutant strain with a linear epitope mutation (E347K) was identified for the first time, and by 2005, NDV strains harboring additional mutations had become predominant.

In view of the foregoing, it is thought that the conventional vaccine strain LaSota may not provide effective protection against currently circulating Newcastle disease viruses. In one example, the vaccine strain KBNP-C4152 (Accession No.: KCTC 10984BP), which exhibits excellent antigenicity and is disclosed in Korean Patent No. 10-0862049-00-00, may be used as a parent strain for the production of the virus with improved thermostability according to the present disclosure.

Another example of the present disclosure provides a method for immunization against Newcastle disease virus, the method comprising the step of administering the Newcastle disease virus, a Newcastle disease vaccine composition, the polypeptide, the polynucleotide, and/or the recombinant vector of the present disclosure to a subject in need thereof.

Still another example of the present disclosure provides a method for preventing infection with Newcastle disease virus, the method comprising the step of administering the Newcastle disease virus, a Newcastle disease vaccine composition, the polypeptide, the polynucleotide, and/or the recombinant vector of the present disclosure to a subject in need thereof.

Yet another example of the present disclosure provides a method for preventing or treating Newcastle disease, the method comprising the step of administering the Newcastle disease virus, a Newcastle disease vaccine composition, the polypeptide, the polynucleotide, and/or the recombinant vector of the present disclosure to a subject in need thereof.

The vaccine composition of the present disclosure can be used to induce an enhanced immune response to a Newcastle disease antigen in a subject (animal), thereby providing a method for preventing or treating Newcastle disease by enhancing the immune response to the Newcastle disease antigen in the anima. Preferably, the disease can be prevented.

The object (animal) may refer to poultry including chickens, pheasants, ducks, geese, turkeys, quails, etc., as well as other animals including humans (primates), dogs, cats, pigs, cows, etc., but is not limited thereto.

The subject (animal) may be a non-human animal, but is not limited thereto.

Specifically, the method may comprise the step of administering the vaccine composition to the subject via subcutaneous injection, intravenous injection, intradermal injection, parenteral injection, intramuscular injection, needle-free injection, electroporation, oral delivery, intranasal delivery, oronasal delivery, or any combination thereof.

The present invention may provide a kit for performing any of the foregoing methods. The kit may comprise a container, preferably containing the vaccine composition provided herein, a pharmaceutically acceptable carrier, an adjuvant, and instructions for use for administering the immunogenic composition to an animal in need thereof to alleviate clinical signs or effects of Newcastle disease infection, preferably to reduce the frequency or severity of Newcastle disease. The kit may also comprise an injection means and/or other administration means. Moreover, the kit may comprise a solvent. The attenuated vaccine may be lyophilized and reconstituted with the solvent to form a solution for injection and/or inhalation. The solvent may be water, saline, a buffer, or an adjuvant-containing solvent.

The kit may comprise separate containers containing an attenuated virus, a solvent, and/or a pharmaceutically acceptable carrier. Instructions for use may be provided in the form of a label affixed to one or more of the containers and/or a printed material.

### Recombinant Newcastle Disease Virus KBNP-C4152

As used herein, the term “velogenic Newcastle disease virus” refers not only to conventionally classified velogenic Newcastle disease viruses, but also to all pathogenic Newcastle disease viruses exhibiting at least moderate pathogenicity, unless otherwise specified. As used herein, the term “velogenic Newcastle disease virus” refers to a virus that exhibits pathogenicity by producing infectious virus particles in all cells of the host upon infection. When the amino acid sequence at positions 113 to 116 of the F protein is represented by the sequence of Chemical Formula 1 below, it can be cleaved by furin or furin-like proteases (hereinafter referred to as “furin”), which are distributed in most cells of the host, thereby forming an active structure that enables the virus to infect host cells. Accordingly, the pathogenic Newcastle disease virus is defined as a virus comprising a nucleotide sequence that encodes the amino acid sequence represented by Chemical Formula 1 below as the coding sequence for positions 113 to 116 of the F protein (where the amino acid positions of the F protein are numbered according to SEQ ID NO: 6):

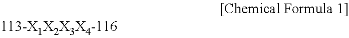

In the above formula,

X₁, X₃ and X₄ are each independently arginine or lysine, and

X₂ is an amino acid selected from the group consisting of alanine, isoleucine, leucine, methionine, phenylalanine, proline, tryptophan, valine, asparagine, cysteine, glutamine, glycine, serine, threonine, tyrosine, aspartic acid, glutamic acid, arginine, histidine, and lysine.

At this time, if the 112^{th} amino acid of the F protein is a basic amino acid such as arginine or lysine, the virus exhibits even higher pathogenicity.

As used herein, the term “lentogenic Newcastle disease virus” refers not only to conventionally classified lentogenic Newcastle disease virus stains, but also to avirulent Newcastle disease virus stains, unless otherwise specified. As used herein, the term “lentogenic Newcastle disease virus” refers to a virus that exhibits low pathogenicity by undergoing localized viral replication, which occurs only when the virus is activated by proteases present in certain cells and in the extracellular regions of the digestive and respiratory tracts. This term refers to the case where the amino acid sequence at positions 113 to 116 of the F protein is represented by the sequence of Chemical Formula 2 below. Accordingly, a lentogenic Newcastle disease virus is defined as a virus comprising a nucleotide sequence that encodes the amino acid sequence represented by Chemical Formula 2 below as the coding sequence for positions 113 to 116 of the F protein (where the amino acid positions of the F protein are numbered according to SEQ ID NO: 6):

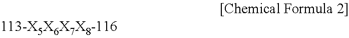

In the above formula,

X₅, X₆ and X₇ are each independently an amino acid selected from the group consisting of alanine, isoleucine, leucine, methionine, phenylalanine, proline, tryptophan, valine, asparagine, cysteine, glutamine, glycine, serine, threonine, tyrosine, aspartic acid, glutamic acid, arginine, histidine, and lysine; X₅ and X₇ are not simultaneously arginine or lysine; and X₈ is arginine or lysine.

The Newcastle disease virus becomes infectious only when the furin cleavage site located in the F protein is cleaved by furin, thereby exposing the fusion peptide region of the F protein that fused with the cell membrane. Since furin is an enzyme distributed throughout the animal body, activation of viral infectivity by furin occurs systemically, resulting in pathogenicity. Accordingly, the pathogenicity of the Newcastle disease virus varies depending on the extent to which the furin cleavage site located in the F protein is recognized and cleaved by furin.

When the furin cleavage site (amino acids at positions 113 to 116) of the F protein of the Newcastle disease virus contains at least three basic amino acids, as represented by the amino acid sequence of Chemical Formula 1 above, the virus exhibits pathogenicity due to the systemic activation of viral infectivity by furin. However, when one or more basic amino acids are substituted with non-basic amino acids, as in the amino acid sequence of Chemical Formula 2 above, recognition and cleavage by furin rarely occur. Instead, cleavage is mediated only by intracellular or extracellular proteases that are locally present, and thus, fatal systemic infection does not occur, resulting in low pathogenicity.

KBNP-C4152 (Accession No.: KCTC 10984BP), which can be used for the production of the Newcastle disease virus with improved thermostability according to the present disclosure, may be produced by a technique for producing a genetically stable attenuated recombinant Newcastle disease virus. This technique is based on the genome of a lentogenic Newcastle disease virus, in which the coding regions for the F protein and the HN protein, both of which are surface antigens, are substituted with those from velogenic Newcastle disease viruses circulating in Korea and other parts of Asia, thereby enhancing protective efficacy against such velogenic viruses. In addition, the 115^{th} codon of the velogenic Newcastle disease virus is substituted with a codon encoding a non-basic amino acid, more specifically, with a codon that requires at least two point mutations to revert to a codon encoding a basic amino acid.

As described above, the recombinant Newcastle disease virus KBNP-C4152 according to the present disclosure exhibits surface antigens identical or similar to those of field strains, and thus has high antigenicity against such field strains and is effectively attenuated. Moreover, it can revert to a velogenic form only after undergoing at least two point mutations at codon 115, and thus possesses excellent characteristics in terms of both stability and safety.

In addition, pathogenic Newcastle disease viruses can be classified into a syncytial type, which forms syncytia, and a granular type, which forms granules, depending on their cytopathic effects. In general, the syncytial type is known to exhibit higher pathogenicity than the granular type.

KBNP-4152 (Accession No.: KCTC 10919BP) according to the present disclosure is characterized in that its pathogenicity is significantly reduced by using a viral clone belonging to the granular type of a velogenic Newcastle disease virus that provides F and HN coding regions. Moreover, with respect to the HN of Newcastle disease virus, velogenic Newcastle disease viruses have a relatively short sequence of only 571 amino acids, whereas lentogenic Newcastle disease viruses have a longer sequence of 577 or 616 amino acids. Accordingly, the C-terminal amino acid sequence of HN can be used to distinguish between velogenic strains and lentogenic strains. Accordingly, KBNP-C4152 according to the present disclosure is a further attenuated recombinant Newcastle disease virus, in which the C-terminus of the HN protein has been modified to correspond to that of a lentogenic strain (577 amino acids).

KBNP-C4152 (Accession No.: KCTC 10984BP) described in the present disclosure is a recombinant Newcastle disease virus comprising coding sequences (CDSs) of the NP, P, M, F, HN, and L genes (see SEQ ID NOs: 3, 4, 5, 6, and 8 for the proteins encoded by these genes), wherein the coding sequences of the NP, P, M, and L genes are derived from a lentogenic Newcastle disease virus, whereas the coding sequences of the F and HN protein are derived from a velogenic Newcastle disease virus, and wherein the codon encoding the 115^{th} amino acid of the F protein of the velogenic Newcastle disease virus is substituted with a codon selected from the group consisting of: alanine-encoding codons GCA, GCC, GCG, and GCU; aspartic acid-encoding codons GAC and GAU; phenylalanine-encoding codons UUC and UUU; isoleucine-encoding codons AUC and AUU; leucine-encoding codons UUA and UUG; serine-encoding codons UCA, UCC, UCG, and UCU; threonine-encoding codons ACC and ACU; valine-encoding codons GUA, GUC, GUG, and GUU; and tyrosine-encoding codons UAC and UAU. The HN gene of the recombinant Newcastle disease virus may be further mutated such that codons 1 to 569 encode amino acids of a velogenic Newcastle disease virus, and codons from position 570 onward encode amino acids of a lentogenic Newcastle disease virus, including the LaSota strain (where the amino acid positions of the HN protein are numbered according to SEQ ID NO: 7).

The recombinant Newcastle disease virus according to the present disclosure may have antigenicity identical or similar to that of a velogenic field strain, due to having surface antigens that are identical or similar to those of the field strain, while exhibiting reduced pathogenicity compared to existing lentogenic vaccine strains. Pathogenic Newcastle disease viruses have a furin cleavage site in the F protein, and the virus becomes infectious when the furin cleavage site is cleaved by furin, thereby exposing the fusion peptide of the F protein that fused with the cell membrane. Since furin is distributed in most cells in the body, systemic infection by the Newcastle disease virus can occur, resulting in high pathogenicity.

In previous attempts to attenuate Newcastle disease virus using reverse genetics, substitution of the 115^{th} amino acid with glycine has been reported. However, in this case, there is a drawback in that pathogenicity can be restored by a single nucleotide mutation in the codon encoding glycine, resulting in substitution with a basic amino acid such as lysine or arginine.

However, the recombinant Newcastle disease virus KBNP-C4152 according to the present disclosure has a codon at the 115^{th} position of the F protein that encodes a non-basic amino acid and requires at least two point mutations to revert to a codon encoding a basic amino acid. Therefore, the likelihood of reversion to pathogenicity is extremely low, and the stability may be significantly improved compared to existing attenuated mutant strains.

In the case where the furin cleavage site of the F protein is modified as described in the present disclosure, cleavage by furin does not occur, and systemic infection does not take place. Instead, the site is cleaved only by trypsin or trypsin-like enzymes that are distributed in certain cells, particularly in the respiratory and digestive tracts, resulting in localized infection only.

In order to achieve such an effect, the 115^{th} codon of the F protein of the present disclosure may be substituted with a codon selected from the group consisting of: alanine-encoding codons GCA, GCC, GCG, and GCU; aspartic acid-encoding codons GAC and GAU; phenylalanine-encoding codons UUC and UUU; isoleucine-encoding codons AUC and AUU; leucine-encoding codons UUA and UUG; serine-encoding codons UCA, UCC, UCG, and UCU; threonine-encoding codons ACC and ACU; valine-encoding codons GUA, GUC, GUG, and GUU; and tyrosine-encoding codons UAC and UAU.

For the production of the recombinant Newcastle disease virus, the HN gene may be further mutated such that codons 1 to 569 encode amino acids of a velogenic Newcastle disease virus, and codons from position 570 onward encode amino acids of a lentogenic Newcastle disease virus, including the LaSota strain.

Newcastle Disease Virus with Improved Thermostability

With respect to the Newcastle disease virus with improved thermostability according to the present disclosure, the LaSota strain, which is used as a conventional vaccine strain, and the recombinant virus KBNP-C4152 (Accession No.: KCTC 10984BP), in which the F and HN surface antigens of a genotype VII Newcastle disease virus are introduced into the LaSota backbone, were subjected to repeated cycles of heat treatment and selection of surviving viruses. Through this process, thermostable LaSota and KBNP-C4152 viruses that exhibit improved thermostability compared to the original viruses were isolated and prepared. Whole-genome sequencing revealed that among the six structural proteins of the ND virus, a single amino acid mutation was identified in each of the genes encoding the P. F, HN, and L proteins.

Specifically, it was confirmed that both the thermostable KBNP-C4152 virus and the thermostable LaSota virus share an identical nucleotide substitution at the same position in the L gene (corresponding to an A745T mutation based on the encoded amino acid). This mutation was found to be associated with the acquisition of thermostability in the virus.

Specifically, the identical nucleotide substitution at the same position within the L gene results in a mutation of the 745^{th} amino acid of the L protein from alanine to threonine, and it was confirmed that this amino acid mutation may contribute to improved thermostability.

The Newcastle disease virus with improved thermostability according to the present disclosure is a Newcastle disease virus comprising NP, P, M, L, F, and HN proteins, wherein the 745^{th} amino acid of the L protein is threonine (the amino acid position of the L protein is numbered according to SEQ ID NO: 8), and the thermostability may be improved compared to that of a Newcastle disease virus in which the 745^{th} amino acid of the L protein is an amino acid other than threonine (e.g., alanine).

In one example, the Newcastle disease virus with improved thermostability may be produced using a KBNP-C4152 virus expression vector as a template, the vector comprising NP, P, M, and L proteins of a lentogenic Newcastle disease virus and F and HN proteins of a velogenic Newcastle disease virus.

In a specific example, the Newcastle disease virus with improved thermostability may comprise the NP protein or its coding gene represented by SEQ ID NO: 3, the P protein or its coding gene represented by SEQ ID NO: 4, the M protein or its coding gene represented by SEQ ID NO: 5, the F protein or its coding gene represented by SEQ ID NO: 6, the HN protein or its coding gene represented by SEQ ID NO: 7, and the L protein or its coding gene represented by SEQ ID NO: 8, and may, for example, comprise a genome represented by SEQ ID NO: 2.

Specifically, a vector may be constructed to introduce a mutation at the 745^{th} amino acid of the L protein from alanine to threonine using overlap extension PCR (OE PCR) technique but is not limited thereto.

In one example, the parental vector pTMH may be prepared according to the structure, characteristics, and production process described in Korean Patent No. 10-0862049-00-00, and a vector introducing the alanine-to-threonine mutation may be inserted into the pTMH backbone to construct plasmid pTMH-CND-745T (see FIG. 8). In addition, three plasmid vectors, pCR-TM-NP, pCR-TM-P, and pCR-TM-L (see FIG. 7), may be prepared as genome transcription vectors for NP, P. and L genes, in which protein expression is initiated by a T7 promoter (see FIG. 7), for the formation of an RNP complex. These plasmids may be transfected into a cell line infected with vaccinia T7 virus to produce a recombinant Newcastle disease virus, referred to as CND-745T.

In one example, RT-PCR was performed to verify the presence of the attenuating marker gene region in the prepared CND-745T virus. As a result, it was confirmed that both CND-745T and KBNP-C4152 have a cleavage site with the sequence structure 112-GRQARL-117. In particular, the alanine (A) at position 115 is a unique feature of the KBNP-C4152 virus, which is not found in wild-type Newcastle disease viruses. These results confirm the presence of the attenuating marker gene region in the CND-745T virus.

In one example, RT-PCR was performed to verify the presence of the thermostability-related marker gene region in the CND-745T virus prepared as described above. As a result, it was confirmed that, unlike KBNP-C4152, the L gene of CND-745T encodes a threonine residue at position 745 of the L protein.

In one example, the KBNP-C4152 virus and the LaSota virus were subjected to heat treatment, and hemagglutination activity and cell infectivity were measured before and after the treatment. In addition, the prepared CND-745T virus and Ulster NDV, a commercial vaccine strain known to exhibit high thermostability, were also evaluated for hemagglutination activity and cell infectivity to compare their thermostability.

Specifically, the cell infectivity of the CND-745T virus was measured to be higher than that of the heat-treated KBNP-C4152 and LaSota viruses, and was found to be comparable to or greater than that of the ulster NDV, thereby confirming its thermostability.

In one example, to analyze the replication capacity of the vaccine strain containing the CND-745T virus, hemagglutination activity was examined, and the viral titer was measured using the Reed-Muench method. In addition, hemagglutination ability was assessed by mixing the vaccine strain with red blood cells, thereby confirming its replication capacity.

Specifically, the viral titer can be measured at a level of 10⁸ EID₅₀/mL or higher, 10^{8.5} EID₅₀/mL or higher, 10⁹ EID₅₀/mL or higher, 10^{9.3} EID₅₀/mL or higher, or 10^{9.5} EID₅₀/mL or higher, but is not limited thereto. Moreover, the viral titer may be measured at a level of 10⁸ TCID₅₀/mL or higher, 10^{8.5} TCID₅₀/mL or higher, 10⁹ TCID₅₀/mL or higher, 10^{9.5} TCID₅₀/mL or higher, 10^{9.7} TCID₅₀/mL or higher, or 10^{9.9} TCID₅₀/mL or higher, but is not limited thereto.

Specifically, the hemagglutination ability may be measured as an HA titer in the range of 27 to 2¹¹, 2⁸ to 2¹⁰, 2^{8.5} to 2^{9.5} or 2^{8.7} to 2^{9.3}, for example, an HA titer of 2⁹, but is not limited thereto.

In one example, the pathogenicity of the vaccine strain containing the CND-745T virus may be evaluated by measuring the embryo mean death time (MDT) over a 7-day period and the Intra cerebral pathic index (ICPI) over a 8-day period, thereby confirming that the strain is non-pathogenic.

Specifically, no mortality was observed during the above period, and thus the virus can be classified as non-pathogenic.

In one example, to evaluate the safety of the vaccine strain containing the CND-745T virus, animals were observed over a 3-week period following ocular inoculation, drinking water inoculation, or no inoculation of the vaccine virus. Body weight and mortality rate were measured to confirm the safety of the vaccine strain.

Specifically, the measurement results showed that the body weights were similar across all inoculation routes, and the mortality rate was 0% in all groups, thereby confirming the safety of the vaccine strain.

In one example, to verify the genetic stability of the vaccine strain containing the CND-745T virus, the virus was serially passaged up to 15 times in specific pathogen-free (SPF) embryonated eggs and up to 5 times in chicks. RT-PCR analysis was then performed to confirm the genetic stability.

Specifically, the nucleotide sequence of the Mlu I site, the nucleotide sequence of the codon at position 115 of the F gene, and the nucleotide sequence of the inserted thermostable region of the L gene were analyzed, and it was confirmed that no mutations had occurred throughout the final passage, thereby confirming genetic stability.

In one example, for serological characterization of the CND-745T virus, a cross-hemagglutination inhibition test (HIT) was performed, and it was confirmed that CND-745T and the donor strain KBNP-C415 exhibited similar results, indicating no serological differences.

In one example, to evaluate the minimum immunogenicity and protective efficacy of the vaccine strain containing the CND-745T virus, the vaccine strain was administered via drinking water inoculation, ocular inoculation, spray inoculation, or not administered, and the hemagglutination inhibition antibody titer was measured in accordance with the method of the World Organisation for Animal Health (OIE). Based on the HIT results, the immune response was evaluated, and protective efficacy was determined by calculating the protection rate based on the mortality rate after challenge inoculation.

Specifically, the highest HIT and the lowest mortality rate were observed in the group subjected to spray inoculation.

In the vaccine composition or method provided in the present disclosure, the content or dosage of the Newcastle disease virus provided in the present disclosure may be an amount of 10^{4.0} EID₅₀ or more, 10^{5.0} EID₅₀ or more, 10^{6.0} EID₅₀ or more, or 10^{7.0} EID₅₀ or more, but is not limited thereto.

The method for preventing Newcastle disease provided in the present disclosure may comprise the step of administering the vaccine composition by at least one method selected from the group consisting of drinking water inoculation, ocular inoculation, and spray inoculation, but is not limited thereto.

The present disclosure provides a method for producing a Newcastle disease virus with improved thermostability, the method comprising the step of inducing a mutation at the 745^{th} amino acid of the L protein to an amino acid other than alanine.

The amino acid other than alanine may be threonine (T), isoleucine (I), leucine (L), lysine (K), methionine (M), phenylalanine (F), tryptophan (W), valine (V), histidine (H), arginine (R), asparagine (N), aspartic acid (D), cysteine (C), selenocysteine (U), glutamic acid (E), glutamine (Q), glycine (G), proline (P), serine (S), or tyrosine (Y), but is not limited thereto.

The step of inducing the mutation may be performed using gene/protein mutagenesis techniques that are commonly employed in the technical field to which the present invention pertains. For example, the mutation may be introduced by appropriate polymerase chain reaction (PCR) (e.g., OE PCR), DNA cleavage and re-ligation (e.g., using restriction enzymes), or mutagenesis (e.g., using an In-Fusion cloning system), but is not limited thereto.

### Advantageous Effects

The Newcastle disease virus provided in the present disclosure and the Newcastle disease virus contained in the Newcastle disease vaccine according to the present disclosure exhibit improved thermostability. Moreover, the Newcastle disease virus that can be produced by a polypeptide comprising an L protein in the Newcastle disease virus, a polynucleotide encoding the polypeptide, or a recombinant vector comprising the polynucleotide also exhibits improved thermostability.

### BRIEF DESCRIPTION OF DRAWINGS

FIGS. 1 and 2 are graphs illustrating results of comparing the hemagglutination ability and cell infectivity of KBNP-C4152 and LaSota before and after heat treatment.

FIGS. 3a and 3b illustrate the results of the nucleotide sequence analysis indicating the amino acid mutations observed after heat treatment of KBNP-C4152 and LaSota.

FIG. 4 illustrates the structure of the constructed parental vector pTMH.

FIG. 5 illustrates the process of introducing an amino acid mutation into the expression vector of the KBNP-C4152 virus using overlap extension PCR (OE PCR).

FIG. 6 illustrates the process of introducing a mutant gene into the L gene using the restriction enzyme Nhe I, and the substitution of the amino acid resulting from the introduction.

FIG. 7 illustrates the structure of the plasmid vectors, pCR-TM-NP, pCR-TM-P, and pCR-TM-L, used in the production of a thermostable recombinant Newcastle disease virus (NDV).

FIG. 8 illustrates the structure of the plasmid pTMH-CND-745T used in the production of a thermostable recombinant NDV.

FIG. 9 illustrates the process of producing a thermostable recombinant NDV by introducing vaccinia T7 virus, plasmid, etc. into cells.

FIG. 10 illustrates the partial genomic structures of LaSota, KBNP-C4152, and CND-745T, as well as the difference in the 745^{th} amino acid in the L protein between the KBNP-C4152 L gene and the CND-745T L gene.

FIG. 11 illustrates the results of hemagglutination assay and selectable marker gene sequence analysis to confirm the presence of the constructed NDV, as well as the nucleotide sequence of the Mlu I site used as a selectable marker for identification of the recombinant virus.

FIG. 12 illustrates the location and nucleotide sequence of the Mlu I restriction enzyme site.

FIG. 13 schematically illustrates the structures of the F1 and F2 regions of CND-745T and the primer binding sites.

FIG. 14 illustrates the results of RT-PCR performed to confirm the presence of an attenuating marker gene in CND-745T.

FIG. 15 illustrates the structure of the cleavage site 112-GGQARL-117 in the CND-745T and KBNP-C4152R2L viruses.

FIG. 16 illustrates the region containing the thermostability-related marker gene region in CND-745T.

FIGS. 17 and 18 are graphs illustrating the results of comparing the hemagglutination ability and cell infectivity of KBNP-C4152, LaSota, and CND-745T before and after heat treatment, along with those of Ulster NDV.

FIGS. 19 to 21 illustrate the results of RT-PCR analysis of nucleotide sequence variations following serial passaging, performed to verify the genetic stability of the CND-745T vaccine strain. In the drawings, “15^{th}” refers to 15 passages in embryonated eggs, and “15^{th}+C5^{th}” refers to an additional 5 passages in 1-day-old chicks after the initial 15 passages.

FIG. 22 illustrates the results of analyzing the serological characteristics of the CND-745T virus.

### MODE FOR CARRYING OUT THE INVENTION

Hereinafter, the present invention will be described in detail with reference to examples. However, the following examples are merely illustrative of the present invention and are not intended to limit the scope of the invention.

### Example 1: Selection and Genetic Characterization of Thermostable Newcastle Disease Virus

### Example 1-1: Selection of Thermostable Newcastle Disease (ND) Virus

Using reverse genetics technology, a recombinant virus, KBNP-C4152 (Accession No.: KCTC 10984BP; name on the certificate of deposit: KBNP-C4152R2L (Newcastle disease virus)), was prepared by grafting the F and HN surface antigens of genotype VII Newcastle disease virus (NDV) onto the backbone of the LaSota vaccine strain (GenBank Accession No.: AY845400). The KBNP-C4152 virus was prepared according to the method described in Korean Patent No. 10-0862049-00-00 (the disclosure of which is incorporated herein by reference). The KBNP-C4152 virus is produced by inserting the F and HN genes of a field velogenic strain (KBNP-4152; KCTC 10919BP) into the LaSota strain, which lacks thermostability. While it serves as an excellent seed virus for inactivated vaccines, its low thermostability may limit its suitability for use as a live vaccine strain administered via spray inoculation.

To overcome this limitation, both the KBNP-C4152 virus and its backbone virus, the LaSota strain, were subjected to repeated cycles of heat treatment at a high temperature (56° C.), followed by selection of only those viruses that survived in specific pathogen-free (SPF) embryonated eggs. As a result, thermostable KBNP-C4152 and LaSota viruses with significantly improved thermostability compared to the original strains were isolated and prepared.

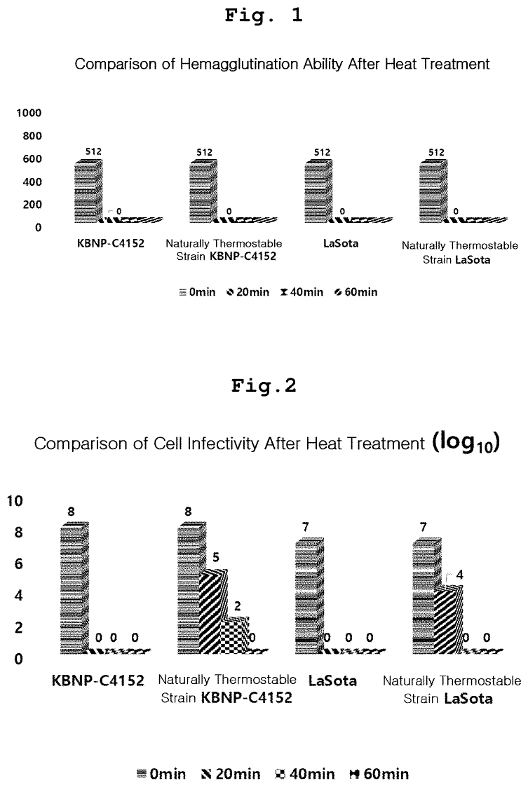

The finally selected thermostable viruses were evaluated for surface protein stability by comparing their hemagglutination ability before and after heat treatment at 56° C., alongside the pre-selection viruses. In addition, acquisition of thermostability was confirmed by assessing their cell infectivity in primary chicken embryo kidney (CEK) cells, which were prepared by treating kidneys from 18- to 19-day-old SPF embryonated eggs with trypsin. The results of the hemagglutination ability and cell infectivity assays are shown in FIG. 1 and FIG. 2, respectively.

Specifically, the hemagglutination ability was evaluated according to the virus hemagglutination assay described in the Veterinary Experimental Manual (National Veterinary Research and Quarantine Service, Ministry of Agriculture and Forestry; Publication Registration Number: 11-1380644-000063-01, see p. 103). The selected thermostable viruses and the pre-selection viruses were subjected to twofold serial dilutions and reacted with an equal volume of 1 (v/v) % chicken red blood cells. After a defined incubation period, the highest dilution exhibiting hemagglutination was determined, and the hemagglutination (HA) titer was measured (see FIG. 1).

In addition, the cell infectivity was assessed by performing tenfold serial dilutions of the selected thermostable viruses and the pre-selection viruses, followed by inoculation of 0.1 mL into primary CEK cells that had been seeded at a density of 10⁴ cells/well in 96-well plates. After incubation for 7 days, the hemagglutination activity of the culture supernatants was evaluated using chicken red blood cells. Hemagglutination of chicken red blood cells was considered positive, and the viral titer was calculated using the Reed-Muench method (see FIG. 2).

As shown in FIGS. 1 and 2, both the selected naturally thermostable KBNP-C4152 and LaSota viruses lost their hemagglutination ability within 10 minutes of heat treatment, similar to the pre-selection viruses (see FIG. 1). However, in terms of cell infectivity in primary CEK cells, while the pre-selection KBNP-C4152 and LaSota viruses retained infectivity only up to 10 minutes after heat treatment, the naturally thermostable KBNP-C4152 strain survived up to 40 minutes and the naturally thermostable LaSota strain up to 20 minutes, exhibiting cytopathic effects. These results indicate that the thermostability of the selected viruses was improved compared to the original strains (see FIG. 2).

### Example 1-2: Genetic Characterization of the Selected Viruses

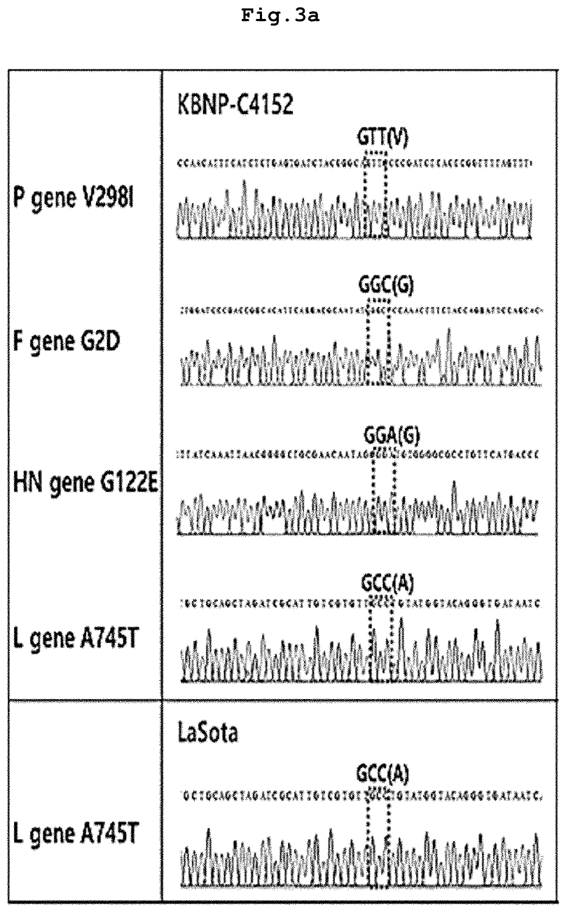

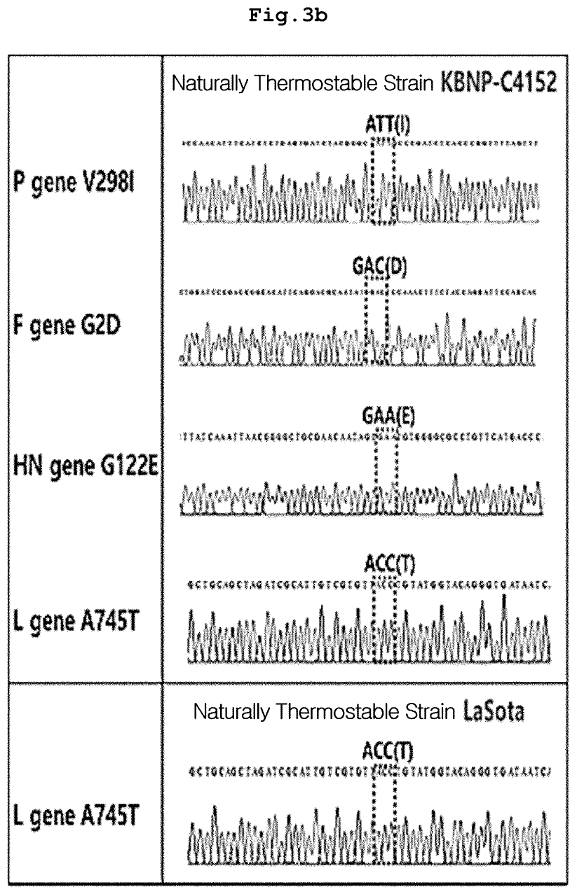

To identify amino acid residues associated with improved thermostability, full-genome sequencing of the viruses before and after selection was outsourced to Bionics, and the results are illustrated in FIGS. 3a and 3b.

As illustrated in FIGS. 3a and 3b, in the case of the naturally thermostable KBNP-C4152 virus, a single amino acid substitution was identified in each of the gene regions encoding the P, F, HN, and L proteins among the six structural proteins of the ND virus, as a result of the virus selection process conducted under stringent experimental conditions. Notably, both the thermostable KBNP-C4152 and LaSota viruses exhibited an identical nucleotide substitution at the same position in the L gene, leading to the same amino acid substitution (A745T), suggesting that this region is associated with the acquisition of thermostability in the virus.

### Example 2: Construction of Vector for Production of Thermostable Recombinant NDV

### Example 2-1: Design and Construction of Parental Vector pTMH for Expression of Thermostable Recombinant NDV

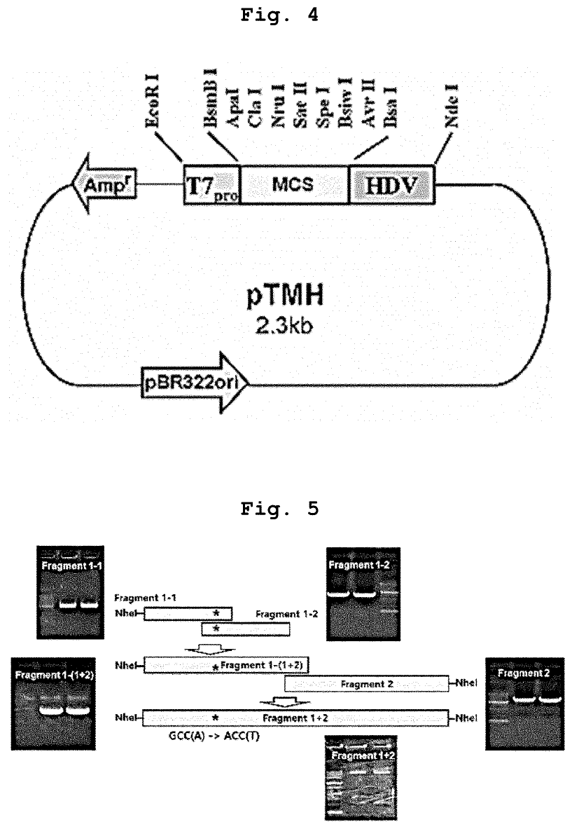

To produce virus particles from NDV cDNA, the viral genome must be transcribed with an identical structure to that of the native genome, without the addition of any extraneous nucleotides at either the 5′ or 3′ end. To achieve this, the parental vector pTMH was prepared. The structure, characteristics, and construction process of the parental vector pTMH are as described in Korean Patent No. 10-0862049-00-00 (the disclosure of which is incorporated herein by reference). The structure and nucleotide sequence of the parental vector pTMH are shown in FIG. 4 and Table 1, respectively.

TABLE 1

| | | SEQ |
|---|---|---|
| Vector | | ID |
| Name | Nucleotide Sequence (5′ → 3′) | NO. |
| pTMH | atcttttactttcaccagcgtttctgggtgagcaaa | 1 |
| | aacaggaaggcaaaatgccgcaaaaaagggaataag | |
| | ggcgacacggaaatgttgaatactcatactcttcct | |
| | ttttcaatattattgaagcatttatcagggttattg | |
| | tctcatgagcggatacatatttgaatgtatttagaa | |
| | aaataaacaaataggggttccgcgcacatttccccg | |
| | aaaagtgccacctgacgtctaagaaaccattattat | |
| | catgacattaacctataaaaataggcgtatcacgag | |
| | gccctttcgtcttcaa | |

### Example 2-2: Construction of Genome Transcription Vector

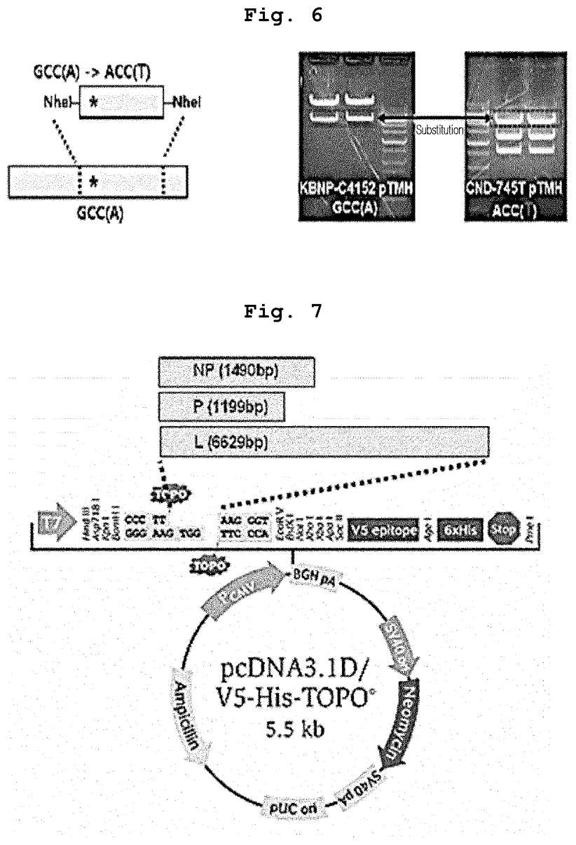

A vector was prepared for the production of a recombinant virus using reverse genetics, in which only the amino acid substitution in the L protein (L745, alanine (A) to threonine (T)) identified as contributing to thermostability was introduced. The remaining structural protein sequences, which are unrelated to thermostability, were retained from the naturally thermostable KBNP-C4152 virus, and this process is illustrated in FIGS. 5 and 6.

To prepare the above-described vector, an amino acid substitution was introduced into the expression vector of the KBNP-C4152 virus using overlap extension PCR (OE PCR). The mutated gene was then inserted into the L gene region using the Nhe I restriction enzyme (see FIG. 5).

More specifically, using the existing KBNP-C4152 virus expression vector as a template, a mutation was introduced at the 745^{th} amino acid residue of the L protein by substituting alanine with threonine through overlap extension PCR (OE-PCR), resulting in the production of fragment 1. Subsequently, fragment 1+2 was produced using OE PCR to contain Nhe I restriction enzyme recognition sites at both ends, and the corresponding region of the KBNP-C4152 virus expression vector was then substituted using the NheI restriction enzyme to construct the final vector. Detailed information on the primers used for the PCR reactions is provided in Table 2 below.

The vector constructed through the above process contains a substitution at the 745^{th} amino acid of the L gene in the KBNP-C4152 virus expression vector, in which alanine was replaced with threonine. Similar to the original KBNP-C4152 expression vector, it uses the safe LaSota virus as a backbone and carries the F and HN antigens of genotype VII NDV, with the 745th amino acid of the L gene substituted with threonine.

TABLE 2

| | | SEQ |
|---|---|---|
| | | ID |
| Primer | Nucleotide Sequence (5′ → 3′ ) | NO. |
| CND- | TATGCTAGCGATGAGTCAACTGTCTTTTAACAGCA | 34 |
| Lgene- | | |
| A745-F | | |
| CND- | TCGCTAGCGTGCTCACCAGACTCTCCGCACAGAAT | 35 |
| Lgene- | | |
| A745-R | | |
| CND- | ATCGCATTGTCGTGTTACCTGCATGGTACAGGGTGA | 36 |
| Lgene- | | |
| A745-IF | | |
| CND- | TCACCCTGTACCATGCAGGTAACACGACAATGCGAT | 37 |
| Lgene- | | |
| A745-IR | | |

### Example 3: Construction and Identification of Thermostable Recombinant NDV

### Example 3-1: Construction of Thermostable Recombinant NDV

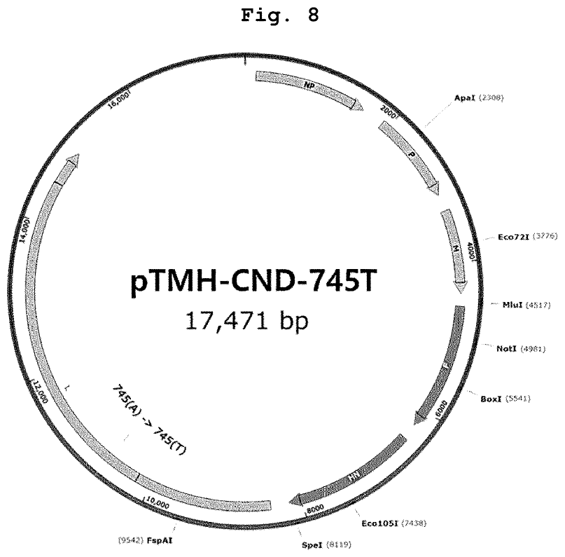

Hep-G2 cell line (ATCC® HB-8065™) was cultured in a 6-well plate at 37° C. under 5% CO₂ until approximately 80% confluence, followed by infection with vaccinia T7 virus, which was kindly provided by Dr. Man-Hoon Park's group at the Mogam Biotechnology Research Institute. To the above-mentioned cell line, three plasmid vectors including pCR-TM-NP, pCR-TM-P, and pCR-TM-L (see FIG. 7) were introduced to express the NP, P, and L proteins required for the production of the NDV RNP complex. These genome transcription vectors are driven by the T7 promoter to initiate protein expression. In addition, a plasmid named pTMH-CND-745T was constructed by inserting the mutated L gene prepared in Example 2-2 into the pTMH vector prepared in Example 2-1. This plasmid enables the production of an accurate and complete full-length chimeric thermostable NDV genome, which is transcribed under the control of the T7 promoter and self-cleaved by the HDV ribozyme (see FIG. 8).

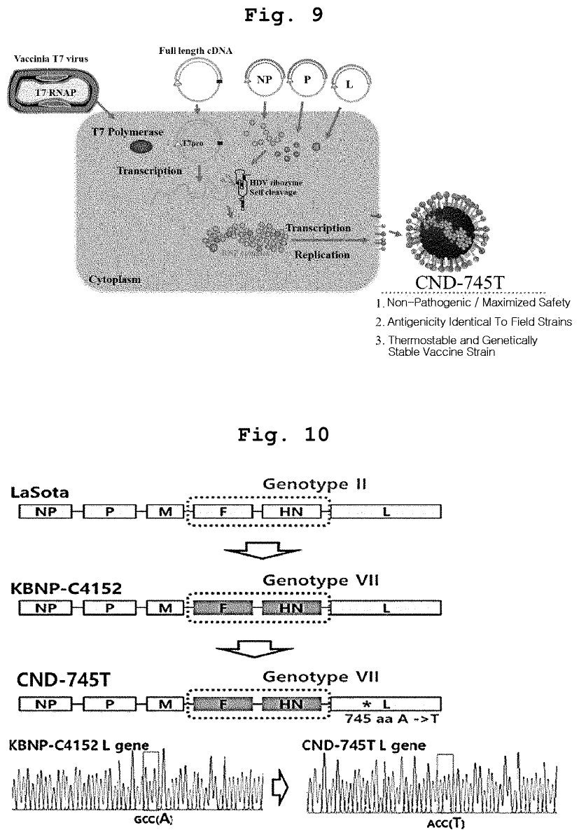

The plasmid vectors were mixed at a ratio of 1:1:0.1:1 and transfected using Lipofectamine™ (Invitrogen Co.). Subsequently, acetylated trypsin was added at a concentration of 1 μg/ml to facilitate the production of a thermostable, non-pathogenic recombinant virus with infectivity, and this process is illustrated in FIG. 9.

The cell line obtained through the above process was cultured at 37° C. for 2 to 3 days. Thereafter, both the cells and the culture supernatant from the 6-well plate were harvested, subjected to three cycles of rapid freezing and thawing, and then inoculated into 11-day-old SPF embryonated eggs. The allantoic fluid was collected to recover the recombinant Newcastle disease virus, which was designated CND-745T. In the transfected cells, a precise copy of the genomic RNA was produced through the combined action of the T7 RNA polymerase promoter and the ribozyme sequence. Simultaneously, the viral helper proteins provided by the transfected expression plasmids enabled subsequent RNA packaging and replication.

The KBNP-C4152 virus was produced by introducing the F and HN genes of genotype VII NDV into the LaSota strain using reverse genetics. The CND-745T virus is structurally identical to the KBNP-C4152 virus, except for a single amino acid substitution at position 745 of the L protein among all structural proteins. The structures of these viruses are shown in FIG. 10, and the complete genomic nucleotide sequence of CND-745T is provided in Table 3.

TABLE 3

| | | SEQ ID |
|---|---|---|
| Strain | Nucleotide Sequence (5′ → 3′) | NO. |
| CND- | MSSVFDEYEQLLAAQTRPNGAHGGGEKGSTLKVDVPVFTLNSDDPEDR | 3 |
| 745T NP | WSFVVFCLRIAVSEDANKPLRQGALISLLCSHSQVMRNHVALAGKQNE | |
| protein | ATLAVLEIDGFANGTPQFNNRSGVSEERAQRFAMIAGSLPRACSNGTP | |
| | FVTAGAEDDAPEDITDTLERILSIQAQVWVTVAKAMTAYETADESETR | |
| | RINKYMQQGRVQKKYILYPVCRSTIQLTIRQSLAVRIFLVSELKRGRN | |
| | TAGGTSTYYNLVGDVDSYIRNTGLTAFFLTLKYGINTKTSALALSSLS | |
| | GDIQKMKQLMRLYRMKGDNAPYMTLLGDSDQMSFAPAEYAQLYSFAMG | |
| | MASVLDKGTGKYQFARDFMSTSFWRLGVEYAQAQGSSINEDMAAELKL | |
| | TPAARRGLAAAAQRVSEETSSIDMPTQQVGVLTGLSEGGSQALQGGSN | |
| | RSQGQPEAGDGETQFLDLMRAVANSMREAPNSAQGTPQSGPPPTPGPS | |
| | QDNDTDWGY | |
| CND- | MATFTDAEIDELFETSGTVIDNIITAQGKPAETVGRSAIPQGKTKVLS | 4 |
| 745T P | AAWEKHGSIQPPASQDNPDRQDRSDKQPSTPEQTTPHDSPPATSADQP | |
| protein | PTQATDEAVDTQLRTGASNSLLLMLDKLSNKSSNAKKGPWSSPQEGNH | |
| | QRPTQQQGSQPSRGNSQERPQNQVKAAPGNQGTDVNTAYHGQWEESQL | |
| | SAGATPHALRSRQSQDNTLVSADHVQPPVDFVQAMMSMMEAISQRVSK | |
| | VDYQLDLVLKQTSSIPMMRSEIQQLKTSVAVMEANLGMMKILDPGCAN | |
| | ISSLSDLRAVARSHPVLVSGPGDPSPYVTQGGEMALNKLSQPVPHPSE | |
| | LIKPATACGPDIGVEKDTVRALIMSRPMHPSSSAKLLSKLDAAGSIEE | |
| | IRKIKRLALNG | |
| CND- | MDSSRTIGLYFDSAHSSSNLLAFPIVLQDTGDGKKQIAPQYRIQRLDL | 5 |
| 745T M | WTDSKEDSVFITTYGFIFQVGNEEATVGMIDDKPKRELLSAAMLCLGS | |
| protein | VPNTGDLIELARACLTMIVTCKKSATNTERMVFSVVQAPQVLQSCRVV | |
| | ANKYSSVNAVKHVKAPEKIPGSGTLEYKVNFVSLTVVPKKDVYKIPAA | |
| | VLKVSGSSLYNLALNVTINVEVDPRSPLVKSLSKSDSGYYANLFLHIG | |
| | LMTTVDRKGKKVTFDKLEKKIRSLDLSVGLSDVLGPSVLVKARGARTK | |
| | LLAPFFSSSGTACYPIANASPQVAKILWSQTACLRSVKIIIQAGTQRA | |
| | VAVTADHEVTSTKLEKGHTLAKYNPFKK | |
| CND- | MGSKLSTRIPAPLMLTTRITLILSCIRPTSSLDGRPLAAAGIVVTGDK | 6 |
| 745T F | AVNVYTSSQTGSIIVKLLPNMPRDKEACAKAPLEAYNRTLTTLLTPLG | |
| protein | DSIRKIQGSVSTSGGGRQARLIGAVIGSVALGVATAAQITAAAALIQA | |
| | NQNAANILRLKESIAATNEAVHEVTDGLSQLSVAVGKMQQFVNDQFNN | |
| | TARELDCIKITQQVGVELNLYLTELTTVFGPQITSPALTQLTIQALYN | |
| | LAGGNMNYLLTKLGIGNNQLSSLIGSGLITGYPILYDSQTQLLGIQVN | |
| | LPSVGNLNNMRATYLETLSVSTTKGYASALVPKVVTQVGSVIEELDTS | |
| | YCIESDLDLYCTRIVTFPMSPGIYSCLSGNTSACMYSKTEGALTTPYM | |
| | ALKGSVIANCKITTCRCTDPPGIISQNYGEAVSLIDRHSCNVLSLDGI | |
| | TLRLSGEFDATYQKNISILDSQVIVTGNLDISTELGNVNNSISNALDS | |
| | LAESNSKLEKINVRLISTSALITYIVLIVISLVFGAFSLGLACYLMYK | |
| | QKAQQKTLLWLGNNTLDQMRATTRA | |
| CND- | MDRAVNRVVLENEEREAKNTWRLVFRIAVLLLMVMTLAISSAALAYST | 7 |
| 745T HN | GASTPHDLASILTVISKTEDKVTSLLSSSQDVIDRIYKQVALESPLAL | |
| protein | LNTESVIMNAITSLSYQINGAANNSGCGAPVHDPDYIGGIGKELIVDD | |
| | ISDVTSFYPSAYQEHLNFIPAPTTGSGCTRIPSFDMSTTHYCYTHNVI | |
| | LSGCRDHSHSHQYLALGVLRTSATGRVFFSTLRSINLDDTQNRKSCSV | |
| | SATPLGCDMLCSKVTGTEEEDYKSVAPTSMVHGRLGFDGQYHEKDLDT | |
| | TVLFKDWVANYPGAGGGSFIDDRVWFPVYGGLKPDSPSDTAQEGKYVI | |
| | YKRHNNTCPDKQDYQIRKAKSSYKPGRFGGKRVQQAILSIKVSTSLGK | |
| | DPVLTIPPNTITLMGAEGRILTVGTSHFLYQRGSSYFSPALLYPMTVN | |
| | NKTATLHSPYTFNAFTRPGSVPCQASARCPNSCITGVYTDPYPLIFHR | |
| | NHTLRGVFGTMLDDEQARLNPVSAVFDNVSRSRVTRVSSSSTKAAYTT | |
| | STCFKVVKTNKTYCLSIAEISNTLFGEFRIVPLLVEILKDDGVREARS | |
| | G | |
| CND- | MASSGPERAEHQIILPESHLSSPLVKHKLLYYWKLTGLPLPDECDFDH | 8 |
| 745T L | LILSRQWKKILESASPDTERMIKLGRAVHQTLNHNSRITGVLHPRCLE | |
| protein | ELANIEVPDSTNKFRKIEKKIQIHNTRYGELFTRLCTHIEKKLLGSSW | |
| | SNNVPRSEEFSSIRTDPAFWFHSKWSTAKFAWLHIKQIQRHLMVAART | |
| | RSAANKLVMLTHKVGQVFVTPELVVVTHTNENKFTCLTQELVLMYADM | |
| | MEGRDMVNIISTTAVHLRSLSEKIDDILRLIDALAKDLGNQVYDVVSL | |
| | MEGFAYGAVQLLEPSGTFAGDFFAFNLQELKDILIGLLPNDIAESVTH | |
| | AIATVFSGLEQNQAAEMLCLLRLWGHPLLESRIAAKAVRSQMCAPKMV | |
| | DFDMILQVLSFFKGTIINGYRKKNAGVWPRVKVDTIYGKVIGQLHADS | |
| | AEISHDIMLREYKSLSALEFEPCIEYDPVTNLSMELKDKAIAHPNDNW | |
| | LASFRRNLLSEDQKKHVKEATSTNRLLIEFLESNDFDPYKEMEYLTTL | |
| | EYLRDDNVAVSYSLKEKEVKVNGRIFAKLIKKLRNCQVMAEGILADQI | |
| | APFFQGNGVIQDSISLIKSMLAMSQLSFNSNKKRITDCKERVSSNRNH | |
| | DPKSKNRRRVATFITTDLQKYCLNWRYQTIKLFAHAINQLMGLPHFFE | |
| | WIHLRLMDTTMFVGDPFNPPSDPTDCDLSRVPNDDIYIVSARGGIEGL | |
| | CQKLWTMISIAAIQLAAARSHCRVTRCARHGNSLYLAEGSGAIMSLLE | |
| | LHVPHETIYYNTLFSNEMNPPQRHFGPTPTQFLNSVVYRNLQAEVTCK | |
| | DGFVQEFRPLWRENTEESDLTSDKAVGYITSAVPYRSVSLLHCDIEIP | |
| | PGSNQSLLDQLAINLSLIAMHSVREGGVVIIKVLYAMGYYFHLLMNLF | |
| | APCSTKGYILSNGYACRGDMECYLVFVMGYLGGPTFVHEVVRMAKTLV | |
| | QRHGTLLSKSDEITLTRLFTSQRQRVTDILSSPLPRLIKYLRKNIDTA | |
| | LIEAGGQPVRPFCAESLVSTLANITQITQIIASHIDTVIRSVIYMEAE | |
| | GDLADTVFLFTPYNLSTDGKKRTSLKQCTRQILEVTILGLRVENLNKI | |
| | GDIISLVLKGMISMEDLIPLRTYLKHSTCPKYLKAVLGITKLKEMFTD | |
| | TSVLYLTRAQQKFYMKTIGNAVKGYYSNCDS | |
| KBNP - | MASSGPERAEHQIILPESHLSSPLVKHKLLYYWKLTGLPLPDECDFDH | 9 |
| C4152 L | LILSRQWKKILESASPDTERMIKLGRAVHQTLNHNSRITGVLHPRCLE | |
| protein | ELANIEVPDSTNKFRKIEKKIQIHNTRYGELFTRLCTHIEKKLLGSSW | |
| | SNNVPRSEEFSSIRTDPAFWFHSKWSTAKFAWLHIKQIQRHLMVAART | |
| | RSAANKLVMLTHKVGQVFVTPELVVVTHTNENKFTCLTQELVLMYADM | |
| | MEGRDMVNIISTTAVHLRSLSEKIDDILRLIDALAKDLGNQVYDVVSL | |
| | MEGFAYGAVQLLEPSGTFAGDFFAFNLQELKDILIGLLPNDIAESVTH | |
| | AIATVFSGLEQNQAAEMLCLLRLWGHPLLESRIAAKAVRSQMCAPKMV | |
| | DFDMILQVLSFFKGTIINGYRKKNAGVWPRVKVDTIYGKVIGQLHADS | |
| | AEISHDIMLREYKSLSALEFEPCIEYDPVTNLSMFLKDKAIAHPNDNW | |
| | LASFRRNLLSEDQKKHVKEATSTNRLLIEFLESNDFDPYKEMEYLTTL | |
| | EYLRDDNVAVSYSLKEKEVKVNGRIFAKLIKKLRNCQVMAEGILADQI | |
| | APFFQGNGVIQDSISLTKSMLAMSQLSFNSNKKRITDCKERVSSNRNH | |
| | DPKSKNRRRVATFITTDLQKYCLNWRYQTIKLFAHAINQLMGLPHFFE | |
| | WIHLRLMDTTMFVGDPFNPPSDPTDCDLSRVPNDDIYIVSARGGIEGL | |
| | CQKLWTMISIAAIQLAAARSHCRVARCARHGNSLYLAEGSGAIMSLLE | |
| | LHVPHETIYYNTLFSNEMNPPQRHFGPTPTQFLNSVVYRNLQAEVTCK | |
| | DGFVQEFRPLWRENTEESDLTSDKAVGYITSAVPYRSVSLLHCDIEIP | |
| | PGSNQSLLDQLAINLSLIAMHSVREGGVVIIKVLYAMGYYFHLLMNLF | |
| | APCSTKGYILSNGYACRGDMECYLVFVMGYLGGPTFVHEVVRMAKTLV | |
| | QRHGTLLSKSDEITLTRLFTSQRQRVTDILSSPLPRLIKYLRKNIDTA | |
| | LIEAGGQPVRPFCAESLVSTLANITQITQIIASHIDTVIRSVIYMEAE | |
| | GDLADTVFLFTPYNLSTDGKKRTSLKQCTRQILEVTILGLRVENLNKI | |
| | GDIISLVLKGMISMEDLIPLRTYLKHSTCPKYLKAVLGITKLKEMFTD | |
| | TSVLYLTRAQQKFYMKTIGNAVKGYYSNCDS | |

(In the above CND-745T sequence, the Nhe I restriction site is underlined, and the mutated amino acid at position 745 of the L protein is indicated in bold. In the KBNP-C4152 L protein sequence, the original amino acid at position 745 of the L protein prior to modification is indicated in bold and underlined.) The CND-745T strain produced as described above was designated as ‘BP-CND-745T’ and was deposited with the Korean Collection for Type Cultures (KCTC), located in Jeongeup-si, Jeollabuk-do, Republic of Korea, on Jan. 19, 2021, under accession number KCTC14453BP.

### Example 3-2: Identification of the Constructed NDV

The transfected cell line prepared in Example 3-1 was cultured at 37° C. for 2 to 3 days and then inoculated into 11-day-old SPF embryonated eggs to prepare infectious NDV. Candling was performed every 24 hours after inoculation to monitor embryo viability. At 72 hours post-inoculation, the eggs were chilled at 4° C., and the allantoic fluid was collected for virus verification.

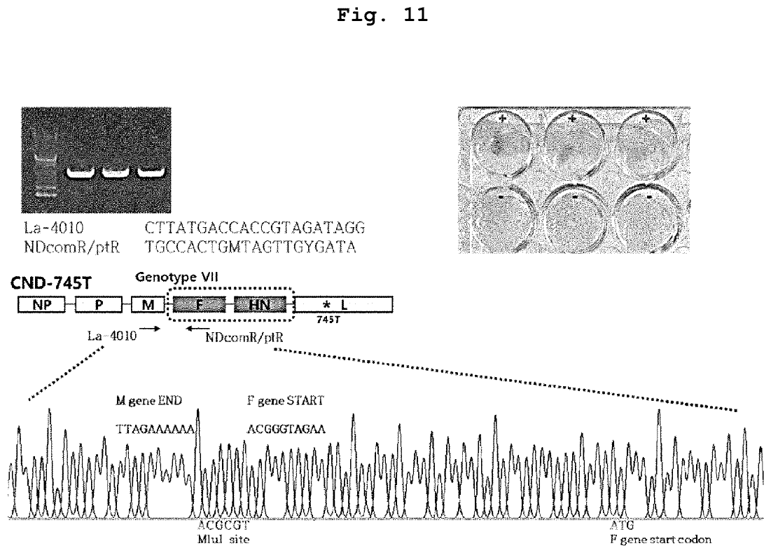

The verification experiment was conducted using substantially the same method as the hemagglutination assay and selectable marker gene sequence analysis described in Example 1-1 to confirm the presence of the recombinant virus. In hemagglutination-positive viruses, the MluI site within the selectable marker gene of the recombinant NDV was identified, and the results are shown in FIG. 11.

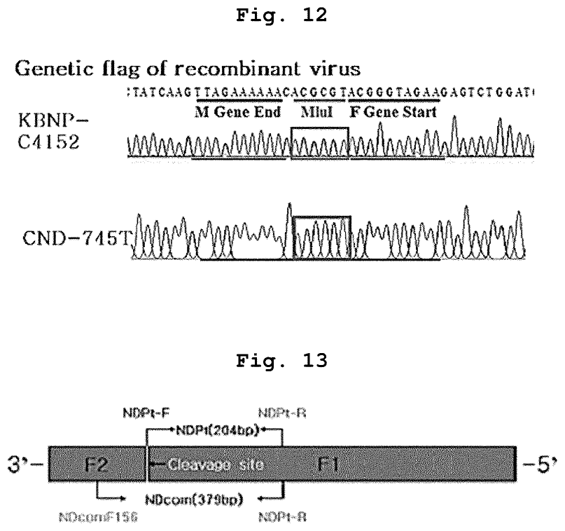

The Mlu I site serves as a genetic marker for identifying the organism as a genetically modified organism. It was introduced into the recombinant NDV as a six-nucleotide MluI restriction enzyme site located immediately upstream of the F gene start codon, and the detailed structure is shown in Table 4 (nucleotide sequence) and FIG. 12.

TABLE 4

| | | SEQ |
|---|---|---|
| | | ID |
| Strain | Nucleotide Sequence (5′ → 3′ ) | NO. |
| Mlu I site | acgcgt | 10 |

### Example 3-3: Identification of Attenuating Marker Gene Region in CND-745T

Except for the amino acid at position 745 of the L gene, CND-745T possesses the same field-type genotype VII F and HN genes as the donor strain KBNP-C4152. However, because the cleavage site was artificially synthesized, the virus is not detected by the velogenic strain-specific primer of the Newcastle disease virus RT-PCR differentiation kit (jointly developed by the National Veterinary Research and Quarantine Service and Intron), which targets the cleavage site of pathogenic NDV strains. Amplification is only possible using a primer that detects all NDV strains.

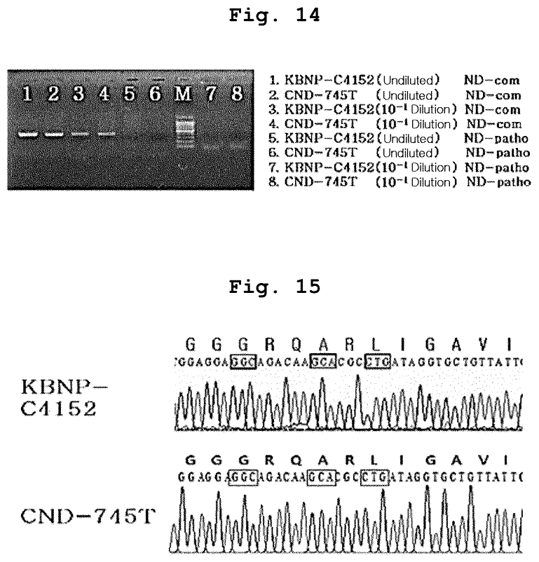

Specifically, RT-PCR was performed using the primer sets listed in Table 5. During the RT-PCR process, the reverse transcription reaction was carried out at 45° C. for 30 minutes. Subsequently, the 3-step cycling was conducted for 40 cycles, consisting of pre-denaturation at 94° C. for 5 minutes; denaturation at 94° C. for 20 seconds, annealing at 50° C. for 30 seconds, and extension at 72° C. for 30 seconds, followed by a final extension at 72° C. for 5 minutes. The structures of the F1 and F2 regions of CND-745T and the primer binding sites are schematically illustrated in FIG. 13, and the results of RT-PCR are shown in FIG. 14.

TABLE 5

| | | SEQ |
|---|---|---|
| | | ID |
| Primer | Nucleotide Sequence (5′ → 3′) | NO. |
| NDPt-F | ggaaggagacrraaacgct | 11 |
| NDPt-R | tgccactgmtagttgygata | 12 |
| NDcomF156 | atacacctortcycagacag | 13 |

Through the RT-PCR, it was confirmed that the cleavage sites of both CND-745T and KBNP-C4152 have the sequence structure 112-GRQARL-117. In particular, the alanine (A) at position 115 is a unique feature of the KBNP-C4152 virus, which is not found in wild-type Newcastle disease viruses, and this sequence structure is illustrated in FIG. 15.

### Example 3-4. Identification of Thermostability-Related Marker Gene Region in CND-745T

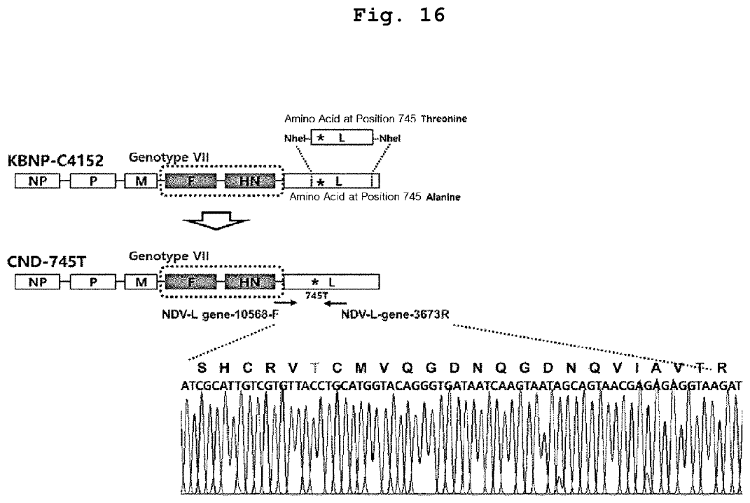

CND-745T can be distinguished from KBNP-C4152 based on a sequence difference at the 745^{th} amino acid of the L gene, which can be identified through nucleotide sequence analysis. Specifically, RT-PCR was performed using the primer sets listed in Table 6. During the RT-PCR process, the reverse transcription reaction was carried out at 45° C. for 30 minutes. Subsequently, the 3-step cycling was conducted for 40 cycles, consisting of pre-denaturation at 95° C. for 15 minutes; denaturation at 94° C. for 20 seconds, annealing at 50° C. for 30 seconds, and extension at 72° C. for 1 minute and 30 seconds, followed by a final extension at 72° C. for 5 minutes, and the results of the analysis are shown in FIG. 16.

TABLE 6

| | | SEQ |
|---|---|---|
| | Nucleotide Sequence | ID |
| Primer | (5′ → 3′) | NO. |
| NDV-L gene-10568-F | tgacatatatattgtcagtgc | 14 |
| NDV-L gene-3673-R | acacggatgatgcccttag | 15 |

As a result, it was confirmed that the amino acid at position 745 of the L gene in CND-745T was threonine, which is different from that of the L gene in KBNP-C4152. This confirmed that a mutation had occurred in the thermostability-related marker gene region in CND-745T.

### Example 4: Identification of Thermostability of the Constructed CND-745T Virus

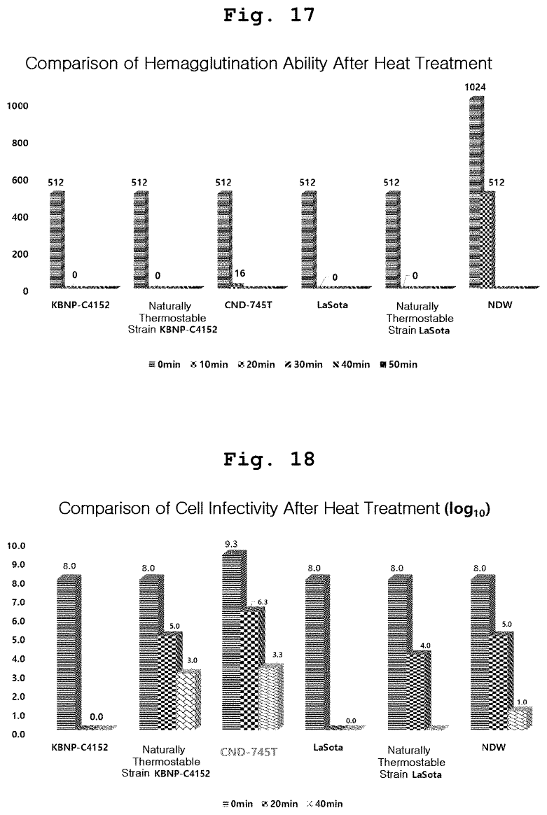

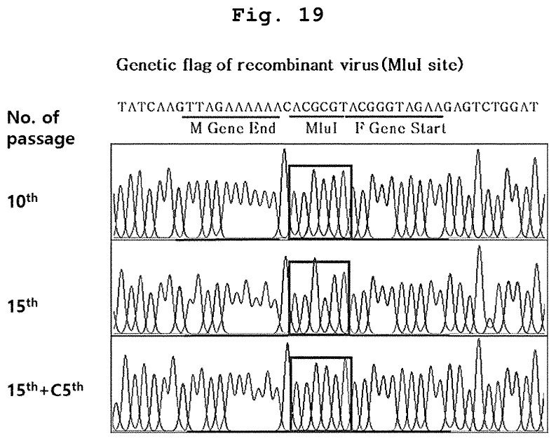

The hemagglutination abilities of the KBNP-C4152 virus, LaSota virus, naturally thermostable KBNP-C4152 virus, and naturally thermostable LaSota virus, which were prepared for the evaluation of hemagglutination ability and cell infectivity in Example 1-1, the CND-745T virus constructed in Example 3-1, and the ulster NDV (Poulvac), a commercially available vaccine strain known for high thermostability, were measured using substantially the same method as described in Example 1-1. In addition, the thermostability was evaluated by assessing the cell infectivity of each virus in primary chicken embryo kidney (CEK) cells, which were prepared by treating kidneys extracted from 18- to 19-day-old SPF embryonated eggs with trypsin, using substantially the same method as in Example 1-1. The results of the hemagglutination activity are shown in FIG. 17, and the results of the cell infectivity assay are shown in FIG. 18.

As shown in FIG. 17, each virus was subjected to heat treatment at 56° C. for 0, 10, 20, 30, 40, or 50 minutes, and the thermal stability of surface proteins was compared based on hemagglutination activity. As a result, no significant difference in hemagglutination-based thermostability was observed between the viruses before and after acquisition of thermostability.

In addition, as shown in FIG. 18, each virus was subjected to heat treatment at 56° C. for 0, 10, 20, 30, or 40 minutes, followed by infection of primary CEK cells. The thermal stability was evaluated by observing cytopathic effects (CPE) in the infected cells. As a result, increased thermostability was observed in the selected viruses (naturally thermostable strains) and the CND-745T virus after heat treatment. Additionally, CND-745T demonstrated thermostability equal to or greater than that of the thermostable Ulster NDV.

### Example 5: Analysis of the CND-745T Vaccine Strain

### Example 5-1: Analysis of Replication Capacity of the CND-745T Vaccine Strain

The CND-745T virus obtained in Example 3-1 was serially diluted tenfold and inoculated at 0.1 mL into primary chicken embryo kidney (CEK) cells, which were prepared by treating kidneys extracted from 18- to 19-day-old SPF embryonated eggs with trypsin and cultured at a density of 10⁴ cells/well in 96-well plates. After incubation for 7 days, the hemagglutination activity of the culture supernatants was evaluated using chicken red blood cells. Hemagglutination of chicken red blood cells was considered positive, and the viral titer was calculated using the Reed-Muench method. As a result, titers of 10^{9.5} EID₅₀/mL or higher and 10^{9.9} TCID₅₀/mL or higher was confirmed, and the results are shown in Table 7.

In addition, CND-745T was serially diluted twofold and mixed with an equal volume of chicken red blood cells. After incubation for a defined period, the hemagglutination titer was evaluated, and a hemagglutination ability of 2⁹ HA titer was confirmed. The results are shown in Table 7.

TABLE 7

| Virus Strain | EID₅₀/ml (log₁₀) | TCID₅₀/ml (log₁₀) | HA titer (log₂) |
|---|---|---|---|
| CND-745T | 9.5 | 9.9 | 9.0 |
| KBNP-C4152 | 9.5 | 9.9 | 9.0 |

As shown in Table 7 above, the viral titer and hemagglutination ability were evaluated, thereby confirming the replication capacity of the CND-745T vaccine strain.

### Example 5-2: Pathogenicity Analysis of the CND-745T Vaccine Strain

To measure the embryo mean death time (MDT), the KBNP-C4152 and CND-745T viruses 10⁻¹ to 10⁻¹⁰, and 0.2 ml of the virus was inoculated into each of five 10-day-old SPF embryonated eggs per group at 9:00 AM and 5:00 PM on the day of inoculation. The eggs were incubated at 37° C. for 7 days and monitored daily. Dead embryos were pre-cooled in a refrigerator at 4° C. for more than 4 hours, followed by an HA test to confirm the presence or absence of viral infection. The MDT value was then calculated according to Equation 1 below.

$$MDT = \{ ( Number of deaths at X hours \times x ) + ( Number of deaths at Y hours \times y ) + ( Number of deaths at X hours \times z ) \} / Total number of deaths \qquad [ Equation 1 ]$$

If the measured index was less than 60 hours, the virus was classified as velogenic; between 60 and 90 hours, as mesogenic; between 90 and 120 hours, as lentogenic; and over 120 hours, as avirulent. To be used as a vaccine virus, the mean death time must be at least 90 hours. Since no embryo deaths were observed during the entire observation period, the measured index was determined to exceed 150 hours, indicating that the virus is avirulent. The results are shown in Table 8 below.

To measure the Intra cerebral pathic index (ICPI), 0.05 ml of each of the two viruses were separately inoculated intracerebrally into ten 1-day-old SPF chicks. The chicks were observed for 8 days to assess pathogenicity. The ICPI was calculated according to Alexander's method (see OIE Terrestrial Manual, 2018; Chapter 3.3.14, pp. 967-968) by scoring each chick daily as follows: 0 for normal, 1 for sick, and 2 for dead. The total daily scores were summed over 8 days and divided by 80. If the measured score was between 0.0 and 0.2, the virus was classified as avirulent; between 0.2 and 0.5, as lentogenic; between 1.0 to 1.5, as mesogenic; and between 1.5 and 2.0, as velogenic. Since no clinical signs or deaths were observed among the chicks during the entire observation period, the intracerebral pathogenicity index (ICPI) was determined to be below 0.1, indicating that the virus was classified as avirulent. The results are presented in Table 8 below.

TABLE 8

| Virus Strain | MDT (h) | ICPI (h) | Pathotype |
|---|---|---|---|
| CND-745T | 150 hr< | <0.1 | Avirulent |
| KBNP-C4152 | 150 hr< | <0.1 | Avirulent |

As shown in Table 8 above, the results of MDT and ICPI measurements confirmed that the CND-745T virus is a safe, avirulent vaccine candidate.

### Example 5-3: Safety Evaluation of the CND-745T Vaccine Strain

To evaluate the safety of the CND-745T virus as a spray vaccine, forty 1-day-old SPF chicks were inoculated with the CND-745T virus using a box-type sprayer (fine spray) available from Three Shine Inc. at a dose of 10^{7.0} EID₅₀ per chick. The chicks were then observed for two weeks for any clinical signs, including respiratory symptoms, depression, diarrhea, or mortality. In addition, to evaluate the safety of the CND-745T virus when administered via drinking water, fifteen 1-day-old SPF chicks were orally inoculated with the virus at a dose of 10^{7.0} EID₅₀ per chick. The chicks were then observed for two weeks for any clinical signs, including respiratory symptoms, depression, diarrhea, or mortality. No clinical signs or mortality were observed in the vaccinated groups in either of the two safety evaluations.

To evaluate the safety of the CND-745T vaccine strain after five or more in vivo passages, forty-five 1-day-old SPF chicks were prepared. Of these, fifteen chicks were assigned to an uninoculated control group, and the remaining thirty chicks were divided into two groups of fifteen each. Each group was inoculated with 10^{6.5} EID₅₀ of the CND-745T (E15K2, Ch5) vaccine virus, which had undergone five passages in 1-week-old or younger SPF chicks, via either ocular or drinking water inoculation. Mortality and changes in body weight were monitored for up to three weeks, and the results are shown in Table 9 below.

TABLE 9

| | | | | | Mortality |
|---|---|---|---|---|---|
| | | | Body Weight | | rate (%) |
| | | Number | 1- | 3- | 21 DPV |
| | | of | Day- | Week- | (day-post- |
| Vaccine | | Chicks | Old | Old | vaccination) |
| CND-745T | Ocular | 15 | 34.6 ± | 154.1 ± | 0 |
| (E15K2, Ch5) | Inoculation | | 1.6 | 17.2 | |
| 10^{6.5} | Drinking | 15 | 35.4 ± | 151.3 ± | 0 |
| EID₅₀/dose | Water | | 1.7 | 19.1 | |
| | Inoculation | | | | |

| | | | | |
|---|---|---|---|---|
| Control Group | 15 | 34.9 ± | 152.2 ± | 0 |

| | |
|---|---|
| 1.7 | 18.8 |

As a result, the body weights of the groups that received ocular and drinking water inoculation were comparable to those of the uninoculated control group, and the mortality rate was 0% in all groups, thereby confirming the safety of the CND-745T vaccine strain.

### Example 5-4: Verification of Genetic Stability of the CND-745T Vaccine Strain

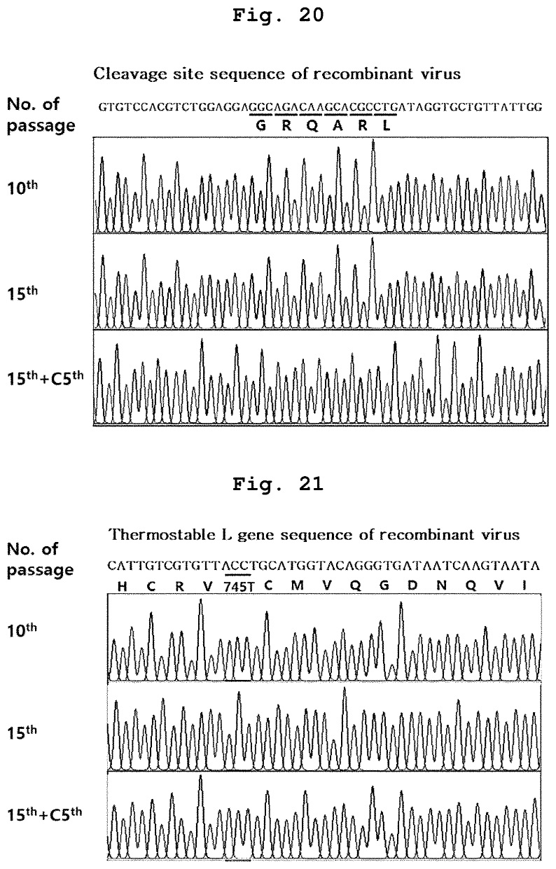

After 15 serial passages in SPF embryonated eggs and 5 additional passages in chicks, RT-PCR was performed using the primer sets listed in Table 10. During the RT-PCR process, the reverse transcription reaction was carried out at 45° C. for 30 minutes for the F, HN, and L proteins of the virus. Subsequently, the 3-step cycling was conducted for 40 cycles, consisting of pre-denaturation at 5° C. for 15 minutes; denaturation at 94° C. for 20 seconds, annealing at 50° C. for 30 seconds, and extension at 72° C. for 2 minutes and 30 seconds, followed by a final extension at 72° C. for 5 minutes. Through the RT-PCR, the nucleotide sequences were analyzed to evaluate the genetic stability of the recombinant CND-745T virus, and the results are shown in FIGS. 19 to 21.

TABLE 10

| | | | Amplicon | SEQ |
|---|---|---|---|---|
| Target | | | Size | ID |
| Region | Primer | Sequence | (bp) | No. |
| M-F | Lasota-P1129F | gatgcagccgggtcgatcg | 2.439 | 16 |
| | LasotaNDVC7d- | aggtggcacgcatattatt | | 17 |
| | Fgene-904R | | | |
| F | NDcom156/f | atacacctcrtcycagacag | 1.499 | 18 |
| | La6203R | acatttttgtagtggcyctcat | | 19 |
| HM | NDV C7d-F-5704-F | tgagcggcaacacatcagc | 1.959 | 20 |
| | SF-7575R | ttaggtggaatagtcagcacc | | 21 |
| HN | NDV-all-HN-737F | ttgtgatatgctgtgctct | 1.041 | 22 |
| | NDV--HN-L- | aagataggtgatacaatg | | 23 |
| | intetgenicR | | | |
| L | NDV C7d-HN-7834- | aggtagtgtcccttgccag | 1.760 | 24 |
| | F | | | |
| | NDV-All-9573-R | tgcgcacatttggctcct | | 25 |
| | NDV-All-9375-F | gatttcttcgcattcaacctg | 1.289 | 26 |
| | NDV-All-10644-R | gctgcagcaagttggattgc | | 27 |
| | NDV-All-10568-F | tgacatatatattgtcagtg | 1.556 | 28 |
| | NDV C7dCND-L- | acacggatgatgcccttag | | 29 |
| | 3673R | | | |
| | NDV C7dCND-L- | atcttccaagcaatataga | 1.762 | 30 |
| | 3512F | | | |
| | NDV C7dCND-L- | gatgccttataccaaga | | 31 |
| | 5373R | | | |
| | NDV C7dCND-L- | attggtgctcgagtgaaag | 1.662 | 32 |
| | 5068F | | | |
| | CND-Trailer-37R | gagttcgaattcgagtccta | | 33 |

Specifically, to verify the genetic stability of the recombinant CND-745T virus with respect to the number of passages in embryonated eggs, the virus was serially passaged 15 times in embryonated eggs. Subsequently, RT-PCR was performed to amplify genomic regions containing the selectable marker Mlu I site (see the 15^{th} passage in FIG. 19), the nucleotide sequence including codon 115 of the F gene (see the 15^{th} passage in FIG. 20), and the introduced thermostable L gene region. Sequence analysis of the amplified products (see the 15^{th} passage in FIG. 21) revealed no nucleotide changes, confirming that the virus is highly genetically stable.

Moreover, to verify the genetic stability of the recombinant CND-745T virus with respect to the number of passages in 1-day-old chicks, 10^{7.0} EID₅₀ of the virus was administered via ocular inoculation to 1-day-old chicks. Five days post-inoculation, the tracheas were harvested and homogenized, and the virus was re-isolated by inoculating embryonated eggs. This procedure was repeated for five sequential passages. The nucleotide sequences of the selectable marker Mlu I site (see the 15^{th}+C5^{th} passage in FIG. 19), codon 115 of the F gene (see the 15^{th}+C5^{th} passage in FIG. 20), and the introduced thermostable L gene region (see the 15^{th}+C5^{th} passage in FIG. 21) were analyzed. The results confirmed that no mutations occurred at all until the final passage, indicating high genetic stability of the recombinant virus.

### Example 5-5: Serological Characterization of the CND-745T Virus

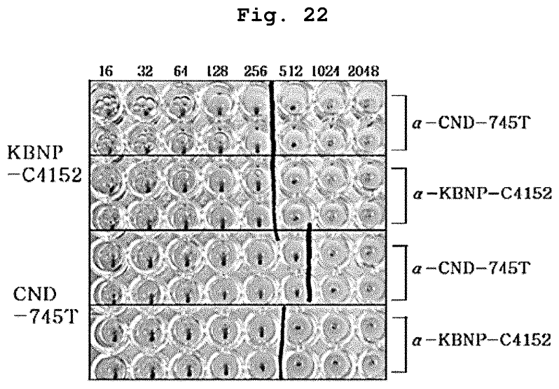

CND-745T was expected to be serologically similar to the donor strain KBNP-C4152, as it retains the same envelope proteins: the fusion (F) protein and the hemagglutinin-neuraminidase (HN) protein. To confirm this, a cross-hemagglutination inhibition test was performed to evaluate it serological characteristics, and the results are shown in FIG. 22 and Table 11 below.

TABLE 11

Antiserum, mean HI titer (log₂)

| Antigen | KBNP-C4152 | CND-745T |
|---|---|---|
| KBNP-C4152 | 9.0 | 9.0 |
| CND-745T | 9.0 | 10.0 |

As expected, CND-745T exhibited similar results to the donor strain KBNP-C4152, confirming that there was no serological difference between the two strains.

### Example 6: Verification of the Minimum Immunogenicity and Protective Efficacy of the CND-745T Vaccine Strain

### Example 6-1: Test Materials

A velogenic Newcastle disease virus (Kr005) was prepared as the challenge virus, and the CND-745T virus obtained in Example 3-1 was used as the test vaccine. A total of 130 one-day-old chicks hatched from SPF eggs imported directly from Charles River (USA) were used in the study. The chicks were divided into three groups of 40 each, based on the route of administration: a drinking water inoculation group, an ocular inoculation group, and a spray inoculation group. An additional group of 10 chicks was assigned to an uninoculated control group.

### Example 6-2: Vaccination

For drinking water inoculation, CND-745T was prepared in 0.1 mL of water at doses of 10^{4.0}, 10^{5.0}, 10^{6.0}, and 10^{7.0} EID₅₀. The chicks in the drinking water inoculation group were divided into four subgroups of 10 each according to the dose and were orally inoculated with 0.1 mL per chick. For ocular inoculation, CND-745T was prepared in 0.03 mL of water at doses of 10^{4.0}, 10^{5.0}, 10^{6.0}, and 10^{7.0} EID₅₀. The chicks in the ocular inoculation group were divided into four subgroups of 10 each according to the dose and were ocularly inoculated with 0.03 mL per chick. For spray inoculation, CND-745T was diluted in 200 ml of water to the doses of 10^{4.0}, 10^{5.0}, 10^{6.0}, and 10^{7.0} EID₅₀, using a cabinet-type sprayer (Samkwang; particle size ≤100 μm). The chicks in the spray inoculation group were divided into four subgroups of 10 each according to the dose and were inoculated once via spray. In addition, a control group of 10 chicks was prepared without any inoculation.

### Example 6-3: Measurement of Hemagglutination Inhibitor Antibody Titers and Evaluation of Protective Efficacy Against NDV

To measure the hemagglutination inhibition (HI) antibody titers against NDV, blood samples were collected two weeks after vaccination according to the procedure described in Example 6-2, and prior to challenge with the virulent virus. The HI antibody titers against CND-745T were measured using the hemagglutination inhibition test (HIT) in accordance with the method of the World Organisation for Animal Health (OIE).

To evaluate the protective efficacy against NDV, the chicks were challenged two weeks after vaccination via intramuscular injection with 2×10^{5.0} EID₅₀/0.1 ml of the Kr005 strain, as described in Example 6-1. The protection rate was calculated based on the mortality rate observed 14 days after the challenge.

### Example 6-4: Test Results

The HIT titers measured in accordance with the method described in Example 6-3 are shown in Table 12 below, and the protective efficacy is shown in Table 13 below.

TABLE 12

| Route of | | |
|---|---|---|
| Administration | Inoculation Dose | HA Ag (CND-745T) |
| Spray Inoculation | 10^{4.0} | 3.7 ± 0.8 |
| | 10^{5.0} | 4.4 ± 0.6 |
| | 10^{6.0} | 4.7 ± 0.5 |
| | 10^{7.0} | 5.4 ± 0.7 |
| Ocular Inoculation | 10^{4.0} | 3.5 ± 0.6 |
| | 10^{5.0} | 4.3 ± 0.9 |
| | 10^{6.0} | 4.8 ± 1.0 |
| | 10^{7.0} | 5.0 ± 0.8 |
| Drinking Water | 10^{4.0} | 3.1 ± 0.9 |
| Inoculation | 10^{5.0} | 3.8 ± 0.7 |
| | 10^{6.0} | 4.1 ± 1.2 |
| | 10^{7.0} | 4.5 ± 0.7 |
| — | Uninoculated Control | 0 |

(In the table above, HA Ag values are presented as mean HI titers±standard deviation in log₂ units.)

TABLE 13

| | | | Cumulative | | |
|---|---|---|---|---|---|
| Route of | | Number | Mortality | Mor- | Pro- |
| Administra- | Inoculation | of | Over 14 | tality | tection |
| tion | Dose (EID₅₀) | Chicks | Days | rate (%) | rate (%) |
| Spray | 10^{4.0} | 10 | 1 | 10 | 90 |
| Inoculation | 10^{5.0} | 10 | 0 | 0 | 100 |
| | 10^{6.0} | 10 | 0 | 0 | 100 |
| | 10^{7.0} | 10 | 0 | 0 | 100 |
| Ocular | 10^{4.0} | 10 | 2 | 20 | 80 |
| Inoculation | 10^{5.0} | 10 | 0 | 0 | 100 |
| | 10^{6.0} | 10 | 1 | 10 | 90 |
| | 10^{7.0} | 10 | 0 | 0 | 100 |
| Drinking | 10^{4.0} | 10 | 4 | 40 | 60 |
| Water | 10^{5.0} | 10 | 1 | 10 | 90 |
| Inoculation | 10^{6.0} | 10 | 0 | 0 | 100 |
| | 10^{7.0} | 10 | 0 | 0 | 100 |
| — | Uninoculated | 10 | 10 | 100 | 0 |
| | Control | | | | |

According to the HI titer results of the CND-745T vaccinated groups, the HA AG levels increased with higher inoculation doses, and overall, the spray-inoculated group showed the highest HA AG values. In addition, the evaluation of protective efficacy against NDV revealed that higher inoculation doses were associated with lower mortality rates, with the spray-inoculated group exhibiting the lowest mortality among all groups.

1. A Newcastle disease virus comprising:
NP, P, M and L proteins of a lentogenic Newcastle disease virus LaSota (AY845400); and
F and HN proteins of a velogenic Newcastle disease virus KBNP-4152 (Accession No.: KCTC 10919BP),
wherein the 115^{th} amino acid of the F protein of the velogenic Newcastle disease virus is an amino acid selected from the group consisting of alanine, aspartic acid, phenylalanine, isoleucine, leucine, serine, threonine, valine, and tyrosine,
wherein the 745^{th} amino acid of the L protein is mutated to an amino acid other than alanine, and
wherein the Newcastle disease virus exhibits improved thermostability compared to a Newcastle disease virus in which the 745^{th} amino acid is alanine.

2. The Newcastle disease virus according to claim 1, wherein the amino acid other than alanine is threonine (T), isoleucine (I), leucine (L), lysine (K), methionine (M), phenylalanine (F), tryptophan (W), valine (V), histidine (H), arginine (R), asparagine (N), aspartic acid (D), cysteine (C), selenocysteine (U), glutamic acid (E), glutamine (Q), glycine (G), proline (P), serine (S), or tyrosine (Y).

3. The Newcastle disease virus according to claim 1, wherein the amino acid other than alanine is threonine (T).

4. The Newcastle disease virus according to claim 1, wherein the HN protein is a recombinant HN protein, in which an amino acid sequence downstream of position 570 of the HN protein of the lentogenic Newcastle disease virus strain LaSota (AY845400) is additionally inserted at the C-terminus of the 569^{th} amino acid of the HN protein of the velogenic Newcastle disease virus KBNP-4152 (Accession No.: KCTC 10919BP).

5. The Newcastle disease virus according to claim 1, comprising a genome represented by the nucleotide sequence of SEQ ID NO: 2.

6. A Newcastle disease vaccine composition comprising the Newcastle disease virus according to claim 1.

7. The Newcastle disease vaccine composition according to claim 6, wherein the vaccine composition is a live vaccine, an inactivated vaccine, a subunit vaccine, a vector vaccine, a chimeric vaccine, or a DNA vaccine.

8. The Newcastle disease vaccine composition according to claim 6, wherein the vaccine composition is administered via an in ovo, intranasal, intratracheal, oral, intradermal, intramuscular, intraperitoneal, intravenous, conjunctival, or subcutaneous route.

9-13. (canceled)

14. A method for preventing infection with Newcastle disease virus, the method comprising the step of administering the vaccine composition according to claim 6 to a subject.

15. The method for preventing infection with Newcastle disease virus according to claim 14, wherein the subject is chicken, pheasant, duck, goose, turkey, or quail.

16-21. (canceled)
